(19) United States
(12) Patent Application Publication
(10) Pub. No.: US 2024/0341675 A1
(43) Pub. Date: Oct. 17, 2024

(54) WOUND MONITORING SYSTEM AND SENSOR THEREOF

(71) Applicant: National University of Singapore, Singapore (SG)

(72) Inventors: John S. Y. Ho, Singapore (SG); Ze Xing, Singapore (SG); Sippanat Achavananthadith, Singapore (SG); Sophie Wan Mei Lian, Singapore (SG)

(21) Appl. No.: 18/682,544

(22) PCT Filed: Aug. 11, 2022
(86) PCT No.: PCT/SG2022/050571

§ 371 Date: Feb. 9, 2024

(30) Foreign Application Priority Data
Aug. 12, 2021 (SG) 10202108822V

(51) Int. Cl. A61B 5/00; A61F 13/00; A61F 13/0203; A61F 13/84; A61L 15/26; A61L 15/62

(52) U.S. Cl. CPC A61B 5/445; A61B 5/002; A61F 13/0213; A61L 15/26; A61L 15/62; A61F 2013/00676; A61F 2013/8473

(57) ABSTRACT

The present disclosure concerns a wound monitoring system for monitoring a bacterial 5 infection at a wound site, comprising a biosensing module that is contactable with the wound site, the biosensing module being configured to output a signal indicative of presence of at least one biomolecule released by bacterial cells at the wound site, and a readout circuitry coupled to the biosensing module for wirelessly transmitting the signal to an external device. The signal is a change in dielectric permittivity of the biosensing module. The present disclosure concerns a method of fabricating the wound monitoring system and a method of monitoring a bacterial infection at the wound.

### TECHNICAL FIELD

The present invention relates, in general terms, to a wound monitoring system for monitoring a bacterial infection at a wound site. The present invention also relates to a method of fabricating the wound monitoring system and a method of monitoring a bacterial infection at the wound.

### BACKGROUND

Wearable biosensors linked with smartphones provide an opportunity to detect pathophysiological events in real-time to notify patients and their caregivers. Such technology has the potential to transform the diagnosis, prevention, and management of chronic medical conditions by enabling continuous monitoring outside of traditional clinical settings. Chronic wounds represent one such condition where management is a major healthcare challenge, consuming over 5% of the healthcare budget. A key factor contributing to the inability of chronic wounds to heal is the presence of pathogenic bacteria, which secrete virulent enzymes that destroy host tissues and disrupt wound recovery. Prompt detection of wound infection is thus critical for clinical intervention to improve patient outcomes. However, current methods for detection either rely on subjective clinical assessments or time-consuming culture-based laboratory tests, leading to delays in timely administration of proper treatment.

Recent advances in flexible electronics have yielded a variety of sensing concepts for interfacing with wounds and tracking clinically-relevant parameters. The most advanced sensors for this purpose measure parameters with well-established methods of electronic transduction, such as temperature, pressure, humidity, and pH. These parameters can afford insights about the local wound environment, but do not directly reflect bacterial virulence. Using optical and electrochemical techniques, biosensors capable of detecting specific markers of pathogenic bacteria at the point-of-care have been developed. However, the integration of such sensors into a wearable device is challenging because of the complexity of the readout instrumentation. Stimuli-responsive materials provide an alternative sensing approach wherein biological signals are transduced into conveniently detectable changes in material properties. Owing to their broadly tunable, tissue-like mechanical properties, stimuli-responsive hydrogels have been widely exploited for sensing applications, such as colorimetric indicators of wound pH. Although recent work demonstrates strategies to extend the programmability and response of hydrogels, existing sensors still lack the ability to detect wound infection and wirelessly transmit data in a way that enables the wound to be continuously monitored without disturbance.

It would be desirable to overcome or ameliorate at least one of the above-described problems.

### SUMMARY

The present invention relates to a sensing system that is based on a flexible, wireless, and battery-free sensor for detecting bacteria virulence. This sensor is based on a DNA hydrogel (DNAgel) that provides a radio-frequency detectable response to deoxyribonuclease (DNase), an enzyme secreted by opportunistic pathogens—such as and including Staphylococcus aureus, Pseudomonas aeruginosa, and Streptococcus pyogenes—commonly implicated in clinical wound infections but not significantly produced by skin-colonizing bacteria. DNase can act as a virulence factor that facilitates bacteria dissemination from biofilms and bacteria evasion of neutrophil extracellular traps deployed by the host immune defense (FIG. 1A). When exposed to extracellular DNase, the DNAgel is degraded via non-specific cleavage of DNA strands, resulting in dissolution of the hydrogel. This changes the dielectric permittivity of the region above an interdigitated electrode, and therefore modulates its capacitance (FIG. 1B). By connecting the electrode to an embedded system, this electronic signal can be read out in a wireless and battery-free manner using near-field communication (NFC), a connectivity technology found on smartphones for short-range communication and wireless power transfer (FIG. 1C). The sensor has a thin and flexible form factor that enables it to be conformally embedded into wound dressings to wirelessly track virulence factor activity on demand (FIG. 1D). We demonstrate the potential of the sensing system for real-time detection of clinically-relevant amounts of S. aureus both in vitro and in a mouse wound model before visible manifestation of infection. This technology may facilitate timely detection of wound infections for improved management of surgical or chronic wounds.

In particular, the invention enables prompt and secure transmission of wireless signals between a smartphone and a wireless wound sensing device that could be interfaced with skin wounds. DNA hydrogel degradation by virulent nuclease secreted by pathogens is sensed via a change of capacitance, which is converted into resonant frequency and signal voltage change. The invention enables battery-free, non-invasive, and in-situ monitoring of invisible wound conditions under wound dressings to provide continuous or on-demand diagnosis and wound information.

The present invention provides a wound monitoring system for monitoring a bacterial infection at a wound site, comprising:
a) a biosensing module that is contactable with the wound site, the biosensing module being configured to output a signal indicative of a presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module.

In some embodiments, the signal is produced by a change in dielectric permittivity of the biosensing module due to degradation of the biosensing module.

In some embodiments, the biosensing module comprises a hydrogel, the hydrogel comprising polynucleotide crosslinked with poly(ethylene glycol) diglycidyl ether (PEGDE).

In some embodiments, the polynucleotide is single stranded DNA and/or single stranded RNA.

In some embodiments, a concentration of polynucleotide in the hydrogel is about 0.02 g/mL to about 0.1 g/mL, and a concentration of PEGDE in the hydrogel is about 0.01 g/mL to about 0.05 g/mL.

In some embodiments, the hydrogel further comprises a dopant selected from poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), silver nanowire (AgNW), gold nanowire or a combination thereof.

In some embodiments, the dopant has a concentration of about 0.1 wt/wt % to about 1 wt/wt % relative to the hydrogel.

In some embodiments, the hydrogel retains more than 80% of its weight at 70% relative humidity after 24 hours.

In some embodiments, the hydrogel has a thickness of about 0.4 mm to about 10 mm.

In some embodiments, the biomolecule is a nuclease.

In some embodiments, the signal indicative of presence of at least one biomolecule released by bacterial cells at the wound site is a change in a dielectric permittivity of the biosensing module of about 0.1 F/m to about 20 F/m.

In some embodiments, the signal is inversely proportional to a degradation of the biosensing module caused by the at least one biomolecule.

In some embodiments, the biosensing module comprises a capacitive sensor.

In some embodiments, the capacitive sensor comprises interdigitated electrodes.

In some embodiments, the electrodes have an inter-electrode spacing between about 30 μm to about 400 μm.

In some embodiments, the change in dielectric permittivity is convertible into a change in capacitance.

In some embodiments, the capacitance is of about 0.4 pF to about 2.5 pF.

In some embodiments, when the hydrogel has a thickness of about 1 mm and the electrodes are spaced apart at about 250 μm, the biosensing module has a capacitance of about 0.15 pF/mm².

In some embodiments, the change in capacitance is convertible into a change in voltage.

In some embodiments, the voltage is of about 0.1 V to about 1 V.

In some embodiments, the readout circuitry comprises a near field communication (NFC) antenna.

In some embodiments, the biosensing module is electrically connected with the readout circuitry.

In some embodiments, a distance between biosensing module and the readout circuitry is between about 2 mm to about 10 mm.

In some embodiments, the bacterial infection is caused by S. aureus, P. aeruginosa, S. pyogenes, Streptococcus agalactiae, Peptostreptococcus anaerobius, Klebsiella pneumonia, Prevotella spp., E. coli, Streptococcus anginosus, E. faecalis, Eikenella corrodens, Morganella morganii, Citrobacter koserior, or a combination thereof.

The present invention also provides a method of fabricating a wound monitoring system for monitoring a bacterial infection at the wound, comprising:
a) configuring a biosensing module to output a signal indicative of a presence of at least one biomolecule released by bacterial cells at the wound site when the biosensing module is contacted with the wound site; and
b) coupling a readout circuitry to the biosensing module for wirelessly transmitting the signal to an external device;
wherein the signal is produced by a change in dielectric permittivity of the biosensing module.

In some embodiments, the method further comprises a step of fabricating the biosensing module, the step comprising crosslinking polynucleotide with poly(ethylene glycol) diglycidyl ether (PEGDE) in order to form a hydrogel for contacting with the wound site.

In some embodiments, the hydrogel is formed at room temperature.

In some embodiments, the hydrogel is formed under a temperature of about 50° C. to about 90° C. for about 1 h to about 4 h.

In some embodiments, the step of fabricating the biosensing module further comprises attaching interdigitated electrodes to the hydrogel.

In some embodiments, the step of fabricating the biosensing module further comprises adhering a protection layer in between the hydrogel and interdigitated electrodes.

The present invention also provides a method of monitoring a bacterial cell concentration of an in vitro sample from a subject, comprising:
a) contacting a biosensing module with the sample, the biosensing module being configured to output a signal indicative of a presence of at least one biomolecule released by bacterial cells in the sample;
b) transmitting the signal from the biosensing module to a readout circuitry for wirelessly transmitting the signal to an external device;
wherein the signal is produced by a change in dielectric permittivity of the biosensing module.

The present invention also provides a method of monitoring a bacterial infection at a wound site, comprising:
a) contacting a biosensing module with the wound site, the biosensing module being configured to output a signal indicative of a presence of at least one biomolecule released by bacterial cells at the wound site; and
b) transmitting the signal from the biosensing module to a readout circuitry for wirelessly transmitting the signal to an external device;
wherein the signal is produced by a change in dielectric permittivity of the biosensing module.

In some embodiments, the change in dielectric permittivity is converted into a change in capacitance before transmitting to the NFC module.

In some embodiments, the change in dielectric permittivity is converted into a change in voltage before transmitting to the NFC module.

### BRIEF DESCRIPTION OF THE DRAWINGS

Embodiments of the present invention will now be described, by way of non-limiting example, with reference to the drawings in which:

FIG. 1 is a schematic representation of embodiments of the present invention;

FIG. 2 shows the hydrogel (DNAgel) synthesis and processability;

FIG. 3 shows the DNAgel bacterial response;

FIG. 4 is a plot of selective degradation of DNAgel across bacteria strains;

FIG. 5 plots dehydration test of DNAgel with wound dressings;

FIG. 6 shows the DNAgel tunability and biocompatibility;

FIG. 7 plots the biocompatibility test of dopants;

FIG. 8 is an example of the WINDOW design and characterization;

FIG. 9 is a schematic for interdigitated sensing electrodes;

FIG. 10 is a circuit analysis of WINDOW;

FIG. 11 is a schematic circuit diagram of WINDOW;

FIG. 12 shows the optimization of coil-coil distance;

FIG. 13 shows the effect of WINDOW orientation on transmission efficiency;

FIG. 14 shows examples of infection detection with WINDOW;

FIG. 15 show a calibration curve of WINDOW sensor; and

FIG. 16 shows the selectivity of DNAgel across bacteria strains.

FIG. 17 shows the stability of hydrogel in PBS.

FIG. 18 shows hydrogel response to patient samples.

### DETAILED DESCRIPTION

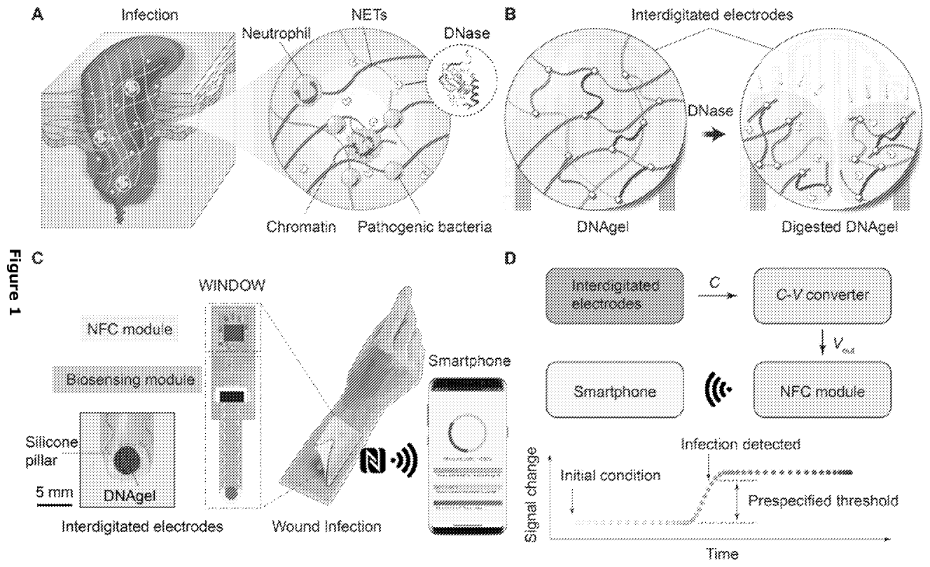

FIG. 1 shows the concept of the monitoring system. (A) Deoxyribonuclease (DNase) is a virulence factor in wound infections. Pathogenic bacteria secrete DNase to evade neutrophil extracellular traps (NETs), which are integral to the host's immune response. (B) Schematic of the infection sensing mechanism. DNAgel is degraded upon exposure to DNase, resulting in a change in the capacitance of the sensor. (C) Schematic of the wireless wound infection monitoring system. The monitoring system integrates the bioresponsive DNAgel in a half-wave-rectified LC biosensing module, and a NFC module to enable smartphone readout of the wound status. Inset image: Sensor-integrated DNAgel stained with Rhodamine B. (D) System block diagram showing signal transduction from the DNAgel-based biosensor to the NFC module and to a smartphone for wireless readout and display.

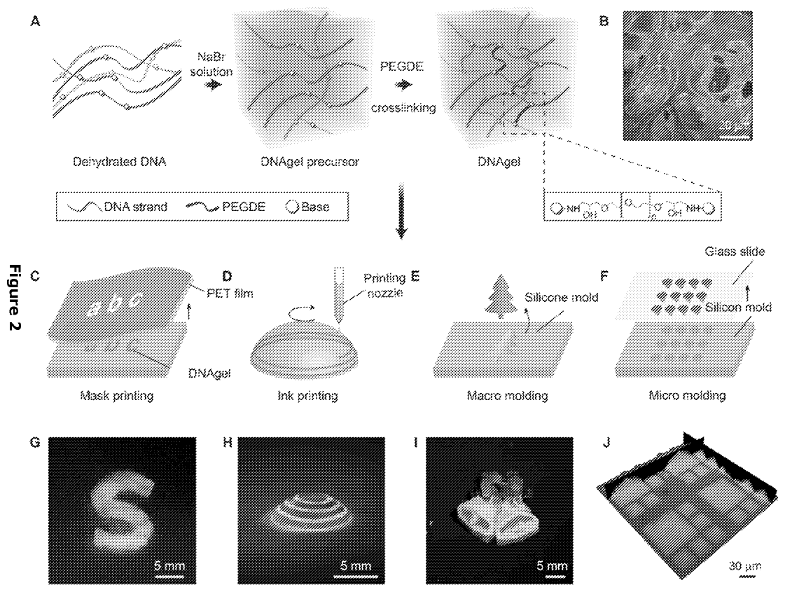

FIG. 2 shows an example of DNAgel synthesis and processability. (A) Schematic of the synthesis process. DNAgel precursor is prepared by dissolving dehydrated DNA strands and then chemically crosslinked by poly(ethylene glycol) diglycidyl ether (PEGDE), forming a 3D network at room temperature. (B) Scanning electron microscope image of freeze-dried DNAgel. (C-F) Strategies for processing DNAgel. DNAgel can be printed on planar (C) and curved surfaces (D) or molded into 3D structures at millimeter (E) to micrometer (F) scales. (G-I) Images of fluorescence-stained DNAgel formed into the letter S (G) printed on a contact lens (H), and macro-molded into a Christmas bell (I). (J) 3D confocal fluorescent image of DNAgel pyramids formed by micro molding. The smallest micro pyramid is ˜10 μm.

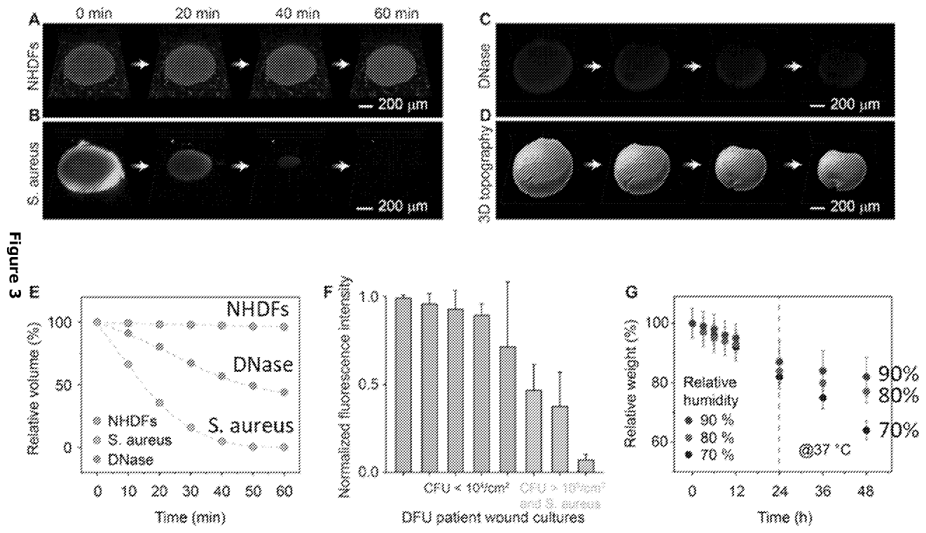

FIG. 3 shows DNAgel bacterial response. (A-C) Confocal fluorescence images of DNAgel co-incubated with live neonatal human dermal fibroblasts (NHDFs, ˜2×10⁴ cells) (A), S. aureus (˜7×10⁷ CFU) (B), and DNase (1 unit/μL) (C). DNAgel is stained using NucBlue (blue). NHDFs are stained using CellMask (purple), and S. aureus using BacLight Kit (green). (D) 3D topographic reconstruction of image in (C). (E) Relative volume change corresponding to the 3D images in (A-C). (F) Fluorescence intensity of DNAgel co-incubated for 24 hours with culture supernatant of wound swab cultures from diabetic foot ulcer (DFU) patients. Error bars show mean±s.d. (n=3 samples). DNAgel is stained using SYBR Gold. (G) Relative weight change of DNAgel under different relative humidity at 37° C. over 48 h.

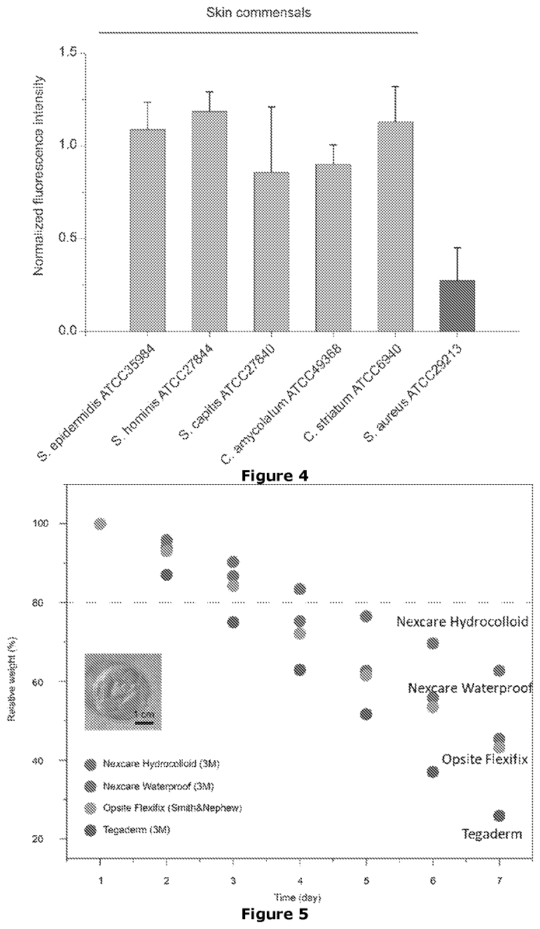

FIG. 4 shows selective degradation of DNAgel across bacteria strains. Fluorescence images of SYBR Gold-stained DNAgel spots were obtained after 24 hours co-incubation with sterile-filtered culture supernatants of Staphylococcus epidermidis ATCC 35984, Staphylococcus hominis ATCC 27844, Staphylococcus capitis ATCC 27840, Corynebacterium amycolatum ATCC 49368 and Staphylococcus aureus ATCC 29213. The fluorescence intensity is quantified by ImageJ by setting thresholds based on positive (DNAse I) and negative (TSB) controls. A higher DNAgel degradation rate is indicated by lower fluorescence intensity. Error bars represent standard deviation from n=3 technical replicates.

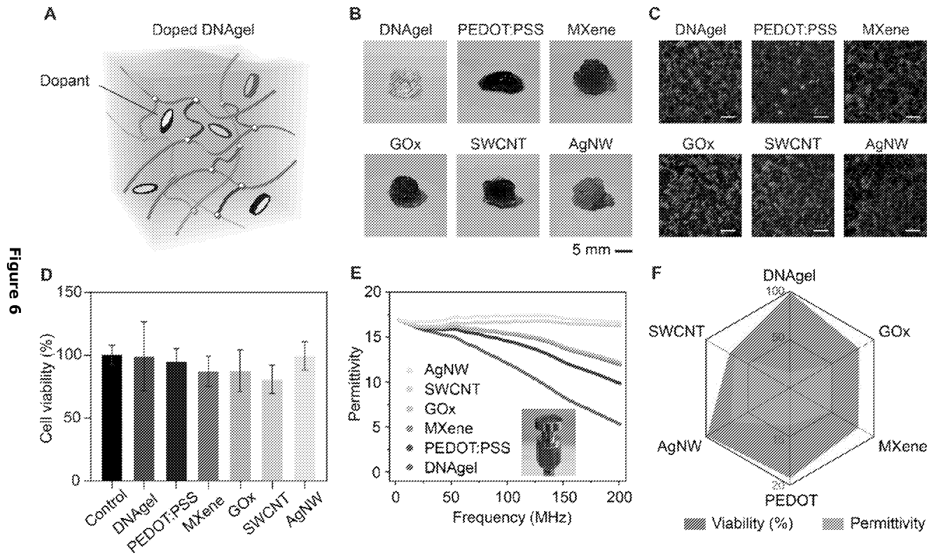

FIG. 5 shows dehydration test of DNAgel with wound dressings. DNAgel was placed in a plastic Petri dish and covered by a series of commercial wound dressings (2.5×2.5 cm²). After the coverage by wound dressings, DNAgel samples were stored at room temperature (˜27° C.) with a relative humidity of ˜70%.

FIG. 6 shows DNAgel tunability and biocompatibility. (A) Illustration of DNAgel with dopant embedded in its 3D network. (B) Images of DNAgels synthesized without dopants, and with poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), and silver nanowire (AgNW). (C) Fluorescent images of NHDFs after 48 h co-incubation with the DNAgels. Scale bars: 500 μm. (D) Viability of NHDFs after 48 h co-incubation with the DNAgels. Error bars show the mean±s.d. (n=3 samples). (E) Dielectric permittivity of DNAgels. Inset shows dielectric probe for permittivity measurement. (F) Radar plot of the viability and permittivity for the DNAgels.

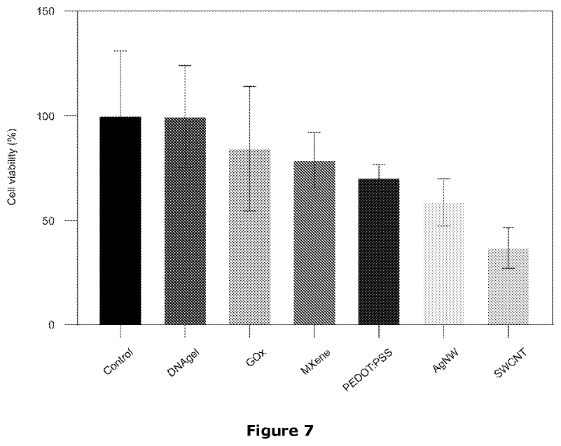

FIG. 7 shows biocompatibility test of dopants. Pristine DNAgel (1 μL) and dopants (1 μL, 1 wt. %) were co-incubated with NHDFs (˜4×10⁴ cells) for 48 h. After the incubation, MTT assay was performed to evaluate the cell viability. Error bars show the mean±s.d. (n=5 samples). Poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), and silver nanowire (AgNW).

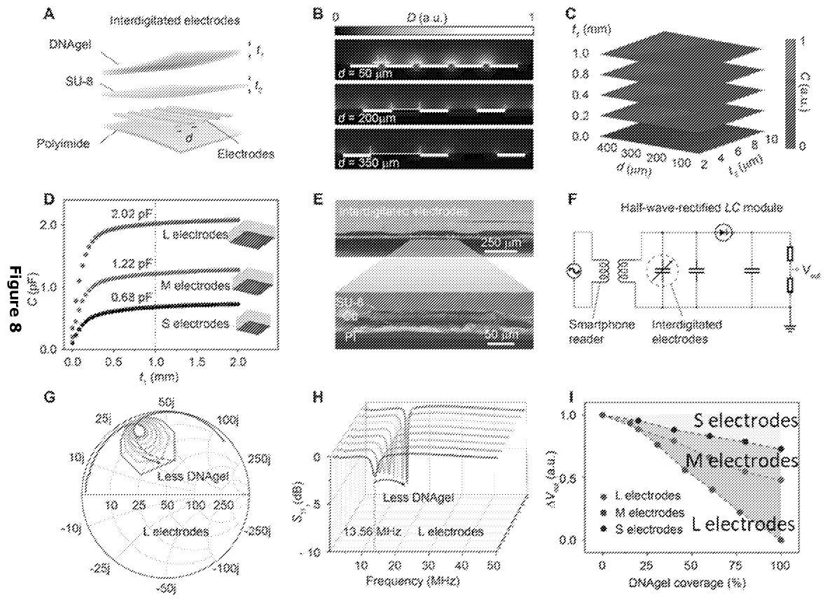

FIG. 8 shows biosensing module design and characterization. (A) Schematic of the capacitive sensing structure comprising an interdigitated electrode pattern with DNAgel in the region of the fringing electric field. The parameters are: the gap between electrodes (d), the DNAgel thickness (t₁), and the SU-8 thickness (t₂). (B) Electric displacement field D for interdigitated electrodes with varying gaps. (C) Contour plots of the capacitance of electrodes as a function of (d, t₁, t₂) normalized to the capacitance with d=50 μm, t₁=1 mm, t₂=2 μm. (D) Capacitance as a function of DNAgel thickness for electrodes with different areas. The areas are: 15 mm² (L), 8 mm² (M), and 3.5 mm² (S) with (d=250 μm, t₂=2 μm). (E) Colored scanning electron microscope image of the cross section of electrodes. (F) Diagram of the circuit of biosensing module. Changes in the capacitive electrodes detune the LC circuit, resulting in a change in the output voltage (Vₒᵤₜ). (G) Smith chart of the LC circuit as the DNAgel coverage decreases from 100% to 0%. The frequency range is from 0.5 to 50 MHz. (H) Reflection coefficient S11 of the LC circuit as the DNAgel coverage decreases from 100% to 0%. (I) Relative change in output voltage of the biosensing modules as a function of DNAgel coverage.

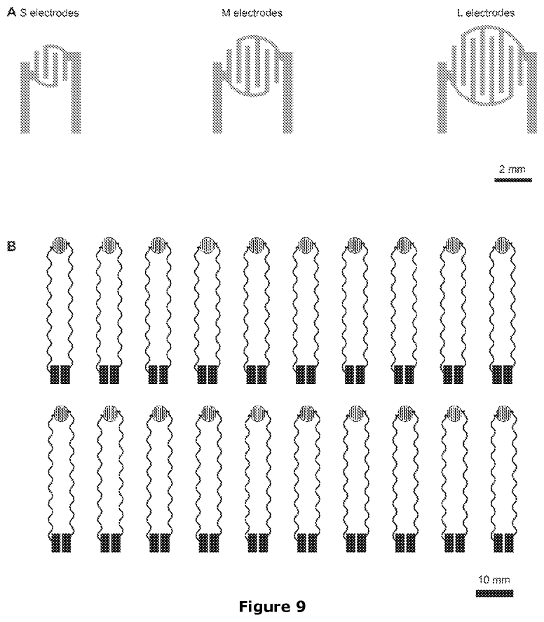

FIG. 9 is a schematic for interdigitated sensing electrodes. (A) Layout of Small (S), medium (M), and large (L) electrodes. (B) Full layout of large electrodes for printing-and-etching process with a designed gap ˜250 μm between electrodes.

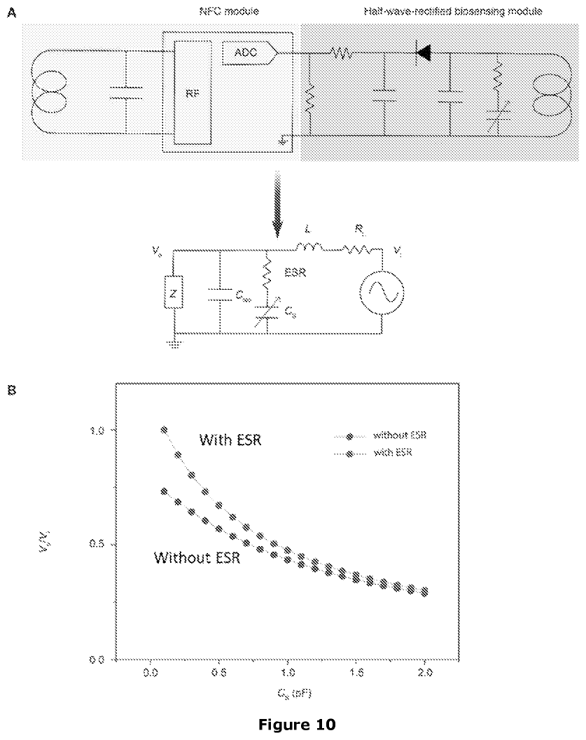

FIG. 10 is a circuit analysis of monitoring system. (A) Circuit diagram of the monitoring system, which consists of a half-wave-rectified biosensing module and a NFC module.

The circuit can be simplified by replacing the rectifier and NFC module with an impedance (Z). (B) Normalized gain (V₀/Vᵢ) of the circuit as a function of sensor capacitance (Eq. S2), with and without considering the loss (ESR) inside the capacitive sensor.

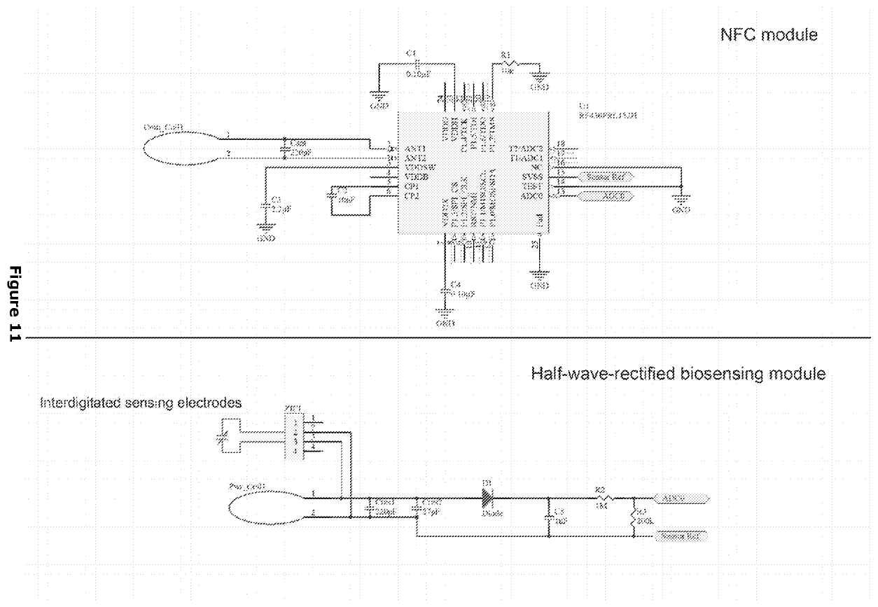

FIG. 11 is a schematic circuit diagram of the monitoring system. The monitoring system consists of a half-wave-rectified biosensing module and a NFC module. Rectified output voltage from biosensing module is cascaded to the ADC in NFC module and transmitted outside to nearby smartphone reader.

FIG. 12 shows optimization of coil-coil distance in the monitoring system. (A) PCB layout of monitoring system. The monitoring system involves two coils for biosensing module and NFC module, respectively. The distance between the two coils is critical to balance footprint and mutual coupling. (B) Model for finite-difference time-domain simulation. (C) Mutual inductance and coupling coefficient (k) with varied coil-coil distance.

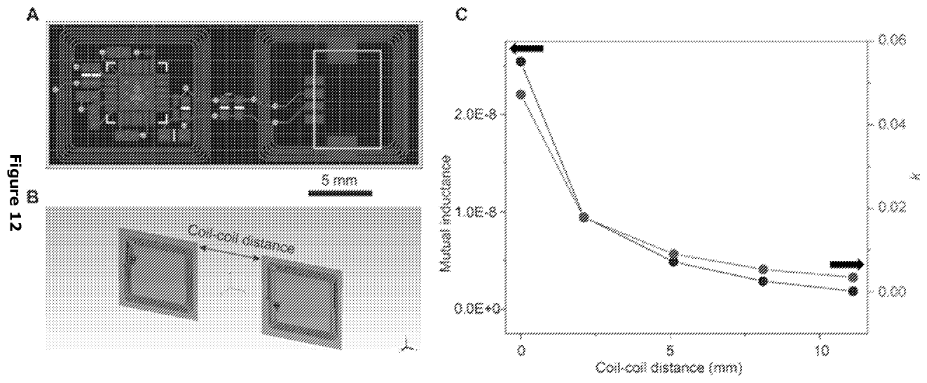

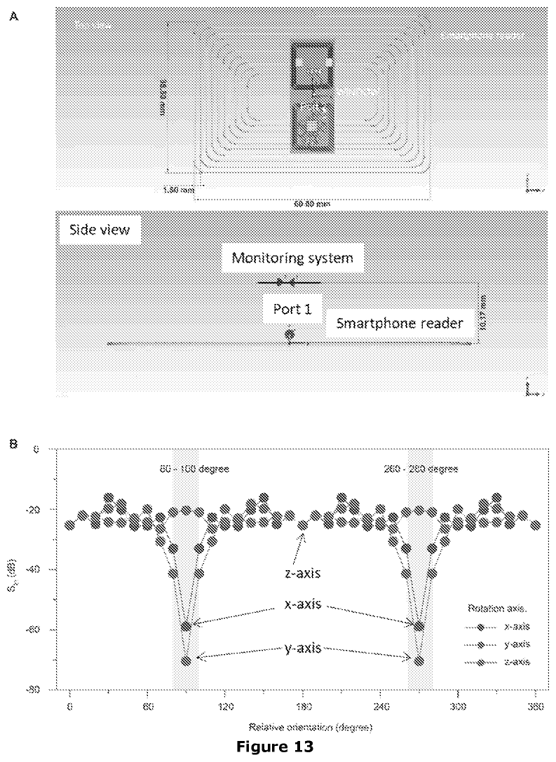

FIG. 13 shows the effect of monitoring system orientation on transmission efficiency. (A) Model for finite-difference time-domain simulation. (B) Transmission coefficient (S₂₁) between the reader antenna (Port 1) and the monitoring system antenna (Port 2). Smaller S₂₁ indicates lower transmission efficiency.

FIG. 14 shows infection detection with the monitoring system. (A) A monitoring system mounted on the index finger. The yellow region shows the readout signal corresponding to the bending angles indicated by the dotted white lines. Inset: Image of the NFC module. (B) Signal readout by a smartphone as the area of the capacitor covered by DNAgel is varied. (C) Signal change when monitoring system is exposed to S. aureus culture supernatant at room temperature over 48 h. Error bars show the mean+s.d. (n=5 data points). (D) Monitoring system mounted on a skin wound in vivo under transparent wound dressing. Left flank wound is used as control. (E) (E) Images of wounds applied with trypic soy broth (TSB) or live S. aureus suspension (10⁵ and 10⁶ CFU) immediately after wounding (0 hour) and 24 hours post wounding. Images are representative from each group (n=2 mice per group). (F) Freely-moving mouse carrying the monitoring system 24 h post-wounding. Dashed line shows the motion trajectory over 30 s (G) S. aureus load at the wound site established by wound culture 24 hours post-wounding. (H) Signal change recorded by for each group. Error bars show the mean±s.d. (n=5 wireless measurements). (I) Smartphone interface displaying signal acquisition and detection of wound infection.

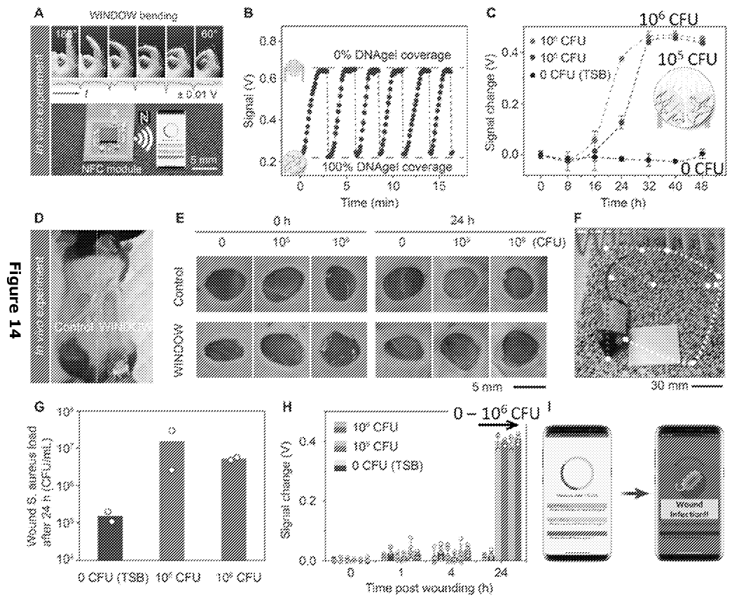

FIG. 15 shows a calibration curve of monitoring system sensor. Signal change as a function of S. aureus CFU number when monitoring system is exposed to S. aureus culture supernatant at room temperature after 24 hours. Error bars show the mean±s.d. (n=5 data points).

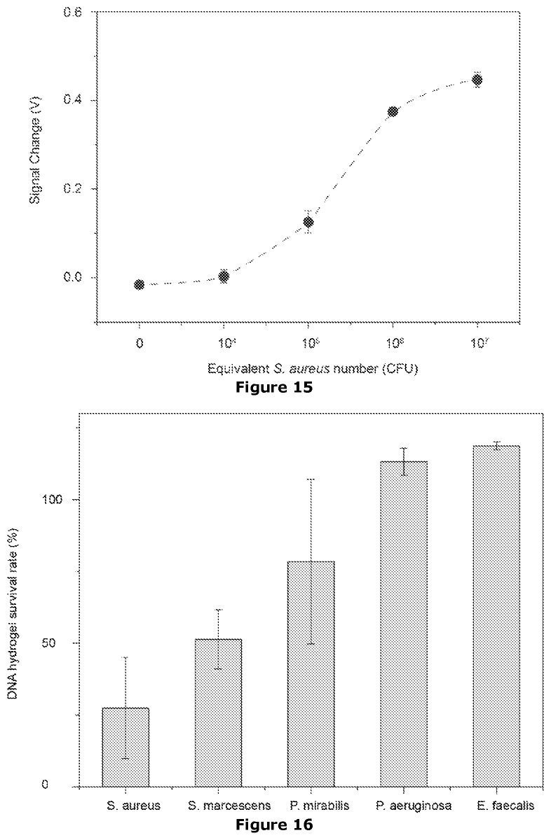

FIG. 16 shows the selectivity of DNAgel across bacteria strains. DNA hydrogel survival rate after co-incubation with supernatants from different bacteria cultures. A lower survival rate indicates a higher reaction activity of DNAgel towards corresponding bacteria. The survival rate is obtained via fluorescent intensity by ImageJ by setting a 30/255 threshold.

As used herein, “device” or “module” refers to a thing or entity made or adapted for a particular purpose, such as a piece of mechanical or electronic equipment. The device can be manually operated, or can be computer implemented with instructions from a software.

As used herein, “system” refers to one or more devices or modules configured with or interacting with each other based on a set of rules. The set of rules can be provided by a software and/or process protocols. In this sense, a system is a group of interacting or interrelated elements that act according to a set of rules to form a unified whole. A system, surrounded and influenced by its environment, is described by its boundaries, structure and purpose and expressed in its functioning.

As used herein, “method” refers to a particular procedure for accomplishing or approaching something. Accordingly, and with reference to this invention, when a particular set of rules is selected, the system can provide a method for monitoring a wound, and in particular, bacterial infection.

The present invention provides a wound monitoring system for monitoring a bacterial infection at a wound site, comprising:
a) a biosensing module that is contactable with the wound site, the biosensing module being configured to output a signal indicative of presence of at least one biomolecule released by bacterial cells at the wound site; and
b) readout circuitry coupled to the biosensing module for wirelessly transmitting the signal to an external device.

The biosensing module undergoes a physical change in the presence of at least one biomolecule released by bacterial cells. This physical change may be translated into a change in a physical property of the biosensing module, which in turn may be used as the signal or used to generate a signal. In this way, a biological signal is converted into an electric signal for monitoring bacterial infection.

In some embodiments, the biosensing module is configured to change its physical property in response to the presence of at least one biomolecule. In other embodiments, the change in physical property is a change in dielectric permittivity. Dielectric permittivity (ε) is the ability of a substance to hold an electrical charge. The dielectric constant (Ka) is the ratio of the permittivity of a substance to vacuum. As the dielectric permittivity is altered, the capacitance of the biosensing module is also modulated. This change can be extracted by the readout circuitry for monitoring the wound.

In some embodiments, the biomolecule is a nuclease. A nuclease is an enzyme capable of cleaving the phosphodiester bonds between nucleotides of nucleic acids. For example, and in some embodiments, the biosensing module changes its physical property as a result of degradation in the presence of the nuclease. In other embodiments, the biomolecule is DNase.

In some embodiments, the biosensing module comprises a hydrogel. In some embodiments, the hydrogel comprises polynucleotide. In some embodiments, the hydrogel comprises polynucleotide crosslinked with poly(ethylene glycol) diglycidyl ether (PEGDE). In some embodiments, the polynucleotide is a single stranded polynucleotide. In some embodiments, the polynucleotide is DNA and/or RNA. In some embodiments, the polynucleotide is single stranded DNA and/or single stranded RNA. The polynucleotide can be cleaved by the biomolecule. This results in degradation of the network structure of the hydrogel, and thus change the physical property of the hydrogel.

In some embodiments, a concentration of polynucleotide in the hydrogel is about 0.02 g/mL to about 0.1 g/mL. In other embodiments, the concentration is about 0.02 g/mL to about 0.09 g/mL, about 0.02 g/mL to about 0.08 g/mL, about 0.02 g/mL to about 0.07 g/mL, about 0.02 g/mL to about 0.06 g/mL, about 0.02 g/mL to about 0.05 g/mL, or about 0.02 g/mL to about 0.04 g/mL.

In some embodiments, a concentration of PEGDE in the hydrogel is about 0.01 g/mL to about 0.05 g/mL. In other embodiments, the concentration is about 0.02 g/mL to about 0.05 g/mL, or about 0.03 g/mL to about 0.05 g/mL.

In some embodiments, the average molecular weight of PEGDE is about 200 to about 1000, about 200 to about 800, about 200 to about 600, or about 400 to about 600. In other embodiments, the average molecular weight of PEGDE is about 500.

In the presence of a nuclease, the hydrogel (comprising polynucleotides) may be degraded via nonspecific cleavage. In some embodiments, the polynucleotide comprises about 5 nucleotides to about 5000 nucleotides, about 10 nucleotides to about 5000 nucleotides, about 15 nucleotides to about 5000 nucleotides, about 20 nucleotides to about 5000 nucleotides, about 25 nucleotides to about 5000 nucleotides, about 30 nucleotides to about 5000 nucleotides, about 40 nucleotides to about 5000 nucleotides, about 50 nucleotides to about 5000 nucleotides, about 60 nucleotides to about 5000 nucleotides, about 70 nucleotides to about 5000 nucleotides, about 80 nucleotides to about 5000 nucleotides, about 90 nucleotides to about 5000 nucleotides, about 100 nucleotides to about 5000 nucleotides, about 150 nucleotides to about 5000 nucleotides, about 200 nucleotides to about 5000 nucleotides, about 400 nucleotides to about 5000 nucleotides, about 500 nucleotides to about 5000 nucleotides, about 800 nucleotides to about 5000 nucleotides, about 1000 nucleotides to about 5000 nucleotides, or about 2000 nucleotides to about 5000 nucleotides. In some embodiments, the polynucleotide comprises about 5 nucleotides to about 500 nucleotides, about 5 nucleotides to about 450 nucleotides, about 5 nucleotides to about 400 nucleotides, about 5 nucleotides to about 350 nucleotides, about 5 nucleotides to about 300 nucleotides, about 5 nucleotides to about 250 nucleotides, about 5 nucleotides to about 200 nucleotides, about 5 nucleotides to about 150 nucleotides, about 5 nucleotides to about 100 nucleotides, about 5 nucleotides to about 90 nucleotides, about 5 nucleotides to about 80 nucleotides, about 5 nucleotides to about 70 nucleotides, about 5 nucleotides to about 60 nucleotides, about 5 nucleotides to about 50 nucleotides, about 5 nucleotides to about 40 nucleotides, about 5 nucleotides to about 30 nucleotides, or about 5 nucleotides to about 20 nucleotides. The length of the polynucleotide may influence the formation of hydrogel network, as well as its physical properties.

In some embodiments, the hydrogel further comprises a dopant. A “dopant” is a component that is added to a material in small amounts, preferably less than 1 wt/wt % relative to the material. In some embodiments, the dopant is an electric conductor. In other embodiments, the dopant is an electrical conductive nanoparticle. In other embodiments, the dopant is characterised by an electrical conductivity of at least about 1500 S/cm, about 1200 S/cm, about 1000 S/cm, about 900 S/cm, or about 800 S/cm. In some embodiments, the dopant is selected from poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), silver nanowire (AgNW), gold nanowire or a combination thereof. A dopant with a high electrical conductivity was found to provide for a greater permittivity response.

In some embodiments, the dopant has a concentration of about 0.1 wt/wt % to about 1 wt/wt % relative to the hydrogel. In other embodiments, the concentration is about 0.1 wt/wt % to about 0.9 wt/wt %, about 0.1 wt/wt % to about 0.8 wt/wt %, about 0.1 wt/wt % to about 0.7 wt/wt %, about 0.1 wt/wt % to about 0.6 wt/wt %, about 0.1 wt/wt % to about 0.5 wt/wt %, about 0.1 wt/wt % to about 0.4 wt/wt %, or about 0.1 wt/wt % to about 0.3 wt/wt %.

In some embodiments, the hydrogel retains more than 80% of its weight at 70% relative humidity after 24 hours. In some embodiments, the hydrogel retains more than 85% or 90% of its weight.

In some embodiments, the hydrogel has a thickness of about 0.4 mm to about 10 mm.

In other embodiments, the thickness is about 0.4 mm to about 9 mm, about 0.4 mm to about 8 mm, about 0.4 mm to about 7 mm, about 0.4 mm to about 6 mm, about 0.4 mm to about 5 mm, about 0.4 mm to about 4 mm, about 0.4 mm to about 3 mm, or about 0.4 mm to about 2 mm. In other embodiments, the thickness is about 1 mm.

In some embodiments, the wound monitoring system for monitoring a bacterial infection at a wound site comprises:
a) a biosensing module that is contactable with the wound site, the biosensing module being configured to output a signal indicative of a presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module.

In some embodiments, the wound monitoring system for monitoring a bacterial infection at a wound site, comprising:
a) a biosensing module that is contactable with the wound site, the biosensing module being configured to output a signal indicative of presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module due to degradation of the biosensing module.

In some embodiments, the wound monitoring system for monitoring a bacterial infection at a wound site, comprising:
a) a biosensing module that is contactable with the wound site, the biosensing module being configured to output a signal indicative of presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module due to degradation of the biosensing module;
wherein the at least one biomolecule is a nuclease; and
wherein the biosensing module comprises a hydrogel of polynucleotide crosslinked with poly(ethylene glycol) diglycidyl ether (PEGDE).

In some embodiments, the signal indicative of presence of at least one biomolecule released by bacterial cells at the wound site is a change in a dielectric permittivity of the biosensing module (or the hydrogel) of about 0.1 F/m to about 20 F/m. In other embodiments, the dielectric permittivity is about 0.5 F/m to about 20 F/m, about 1 F/m to about 20 F/m, about 2 F/m to about 20 F/m, about 4 F/m to about 20 F/m, about 5 F/m to about 20 F/m, about 8 F/m to about 20 F/m, about 10 F/m to about 20 F/m, about 12 F/m to about 20 F/m, or about 14 F/m to about 20 F/m.

In some embodiments, the signal is inversely proportional to a degradation of the biosensing module (or hydrogel) caused by the at least one biomolecule. In particular, the dielectric permittivity may decrease proportionally as the degradation of the hydrogel progresses (correlating to no or low concentration of biomolecule to high concentration of biomolecule). This improves the accuracy of the monitoring system when bacterial infection is low or minimal.

Accordingly, in some embodiments, the wound monitoring system for monitoring a bacterial infection at a wound site, comprising:
a) a biosensing module that is contactable with the wound site, the biosensing module being configured to output a signal indicative of presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module due to degradation of the biosensing module; and
wherein the signal is inversely proportional to a degradation of the biosensing module caused by the at least one biomolecule.

In some embodiments, the biosensing module comprises a capacitive sensor. This allows the dielectric permittivity of the biosensing module to be converted into a capacitance. This capacitance is subsequently converted into a capacitance in the readout circuitry. The change in capacitance can be used as the signal. In some embodiments, the dielectric permittivity is convertible into a capacitance. The biosensing module may have an initial capacitance that may decrease upon exposure to at least one biomolecule and upon degradation of the hydrogel. In some embodiments, the change in dielectric permittivity is convertible into a change in capacitance. In some embodiments, the signal is a change in capacitance of the biosensing module. The change in capacitance is derivable from a change in dielectric permittivity.

In some embodiments, the capacitive sensor comprises interdigitated electrodes. The interdigitated electrodes can be S electrodes, M electrodes, or L electrodes as shown in FIG. 9. In some embodiments, the electrodes have an inter-electrode spacing between about 30 μm to about 400 μm. In other embodiments, the spacing is about 50 μm to about 400 μm, about 70 μm to about 400 μm, about 80 μm to about 400 μm, about 100 μm to about 400 μm, about 120 μm to about 400 μm, about 140 μm to about 400 μm, about 160 μm to about 400 μm, about 180 μm to about 400 μm, or about 200 μm to about 400 μm. In other embodiments, the spacing is about 250 μm.

In some embodiments, the capacitance is of about 0.4 pF to about 2.5 pF. In other embodiments, the capacitance is about 0.5 pF to about 2.5 pF, about 0.6 pF to about 2.5 pF, about 0.7 pF to about 2.5 pF, about 0.8 pF to about 2.5 pF, about 0.9 pF to about 2.5 pF, or about 1 pF to about 2.5 pF.

In some embodiments, the change in capacitance is about 0.4 pF to about 2.5 pF. In other embodiments, the change in capacitance is about 0.5 pF to about 2.5 pF, about 0.6 pF to about 2.5 pF, about 0.7 pF to about 2.5 pF, about 0.8 pF to about 2.5 pF, about 0.9 pF to about 2.5 pF, or about 1 pF to about 2.5 pF.

In some embodiments, when the hydrogel has a thickness of about 1 mm and the electrodes are spaced apart at about 250 μm, the biosensing module has a capacitance of about 0.15 pF/mm².

In some embodiments, the capacitance is convertible into a voltage. In this regard, the capacitance in the biosensing module is converted into a voltage in the readout circuitry. In some embodiments, the dielectric permittivity is convertible into a voltage. In this sense, the voltage is derivable from the dielectric permittivity. For example, instead of converting the dielectric permittivity into a capacitance, the dielectric permittivity may be converted into a voltage in the biosensing module. In some embodiments, the voltage is of about 0.1 V to about 1 V, about 0.2 V to about 1 V, about 0.3 V to about 1 V, about 0.4 V to about 1 V, or about 0.5 V to about 1 V.

The change in voltage may be used as the signal. In some embodiments, the change in voltage is about 0.1 V to about 1 V, about 0.2 V to about 1 V, about 0.3 V to about 1 V, about 0.4 V to about 1 V, or about 0.5 V to about 1 V.

In some embodiments, the biosensing module further comprises a protective coating or layer. The protective coating may be sandwiched between the hydrogel and the electrodes. The protective coating can be a photoresistive coating such as SU-8. In some embodiments, the protective coating has a thickness of about 1 μm to about 3 μm, or preferably about 2 μm.

In some embodiments, the biosensing module further comprises a sensor. The sensor can be for measuring wound temperature, moisture, pH and/or specific biomarkers.

The biosensing module can be a half-wave-rectified biosensing module. A half wave rectifier is defined as a type of rectifier that only allows one half-cycle of an AC voltage waveform to pass, blocking the other half-cycle. Half-wave rectifiers are used to convert AC voltage to DC voltage. The half-wave rectified circuit converts the received RF signal (˜13.56 MHz) into a quasi-DC signal that reflects the status of hydrogel coverage and can be read out by the NFC module.

The readout circuitry includes the NFC module as disclosed herein. In some embodiments, the readout circuitry comprises a near field communication (NFC) antenna.

In some embodiments, the biosensing module is directly and electrically connected to the readout circuitry. Both of them may be inductively powered by an external smartphone, although the signal communication may be through the readout circuitry only.

In some embodiments, the biosensing module is inductively coupled with the readout circuitry. Two conductors are said to be inductively coupled or magnetically coupled when they are configured in a way such that change in current through one wire induces a voltage across the ends of the other wire through electromagnetic induction. In this way, the signal can be sent to wirelessly to a receiver such as a smartphone.

In some embodiments, a distance between biosensing module and the readout circuitry is between about 2 mm to about 10 mm. In other embodiments, the distance is about 2 mm to about 9 mm, about 2 mm to about 8 mm, about 2 mm to about 7 mm, about 2 mm to about 6 mm, about 2 mm to about 5 mm, about 2 mm to about 4 mm. In other embodiments, the distance is less than about 10 mm, about 9 mm, about 8 mm, about 7 mm, about 6 mm, about 5 mm, or about 4 mm.

In some embodiments, the bacterial infection is caused by S. aureus, P. aeruginosa, S. pyogenes, Streptococcus agalactiae, Peptostreptococcus anaerobius, Klebsiella pneumonia, Prevotella spp., E. coli, Streptococcus anginosus, E. faecalis, Eikenella corrodens, Morganella morganii, Citrobacter koserior, or a combination thereof.

In some embodiments, the readout circuitry further comprises a battery. The battery is for powering the sensor (if present). The battery may be chargable via wireless charging or energy harvesting.

In some embodiments, the wound monitoring system further comprises a receiver or an external device. The external device is for wirelessly receiving the signal from the readout circuitry. The external device can be a computing device or a smartphone.

The present invention also provides a method of fabricating a wound monitoring system for monitoring a bacterial infection at the wound, comprising:
a) configuring a biosensing module to output a signal indicative of a presence of at least one biomolecule released by bacterial cells at the wound site when the biosensing module is contacted with the wound site; and
b) coupling a readout circuitry to the biosensing module for wirelessly transmitting the signal to an external device.

In some embodiments, the method of fabricating a wound monitoring system for monitoring a bacterial infection at the wound comprising:
a) configuring a biosensing module to output a signal indicative of a presence of at least one biomolecule released by bacterial cells at the wound site when the biosensing module is contacted with the wound site; and
b) coupling a readout circuitry to the biosensing module for wirelessly transmitting the signal to an external device;
wherein the signal is produced by a change in dielectric permittivity of the biosensing module.

The readout circuitry may be coupled to the biosensing module via electrical connections, or via inductive coupling.

In some embodiments, the signal is a change in capacitance, the change in capacitance is derived from a change in dielectric permittivity of the biosensing module. In some embodiments, the signal is a change in voltage, the change in voltage is derived from a change in dielectric permittivity of the biosensing module.

In some embodiments, the method further comprises a step of fabricating the biosensing module, the step comprising crosslinking polynucleotide with poly(ethylene glycol) diglycidyl ether (PEGDE) in order to form a hydrogel for contacting with the wound site.

In some embodiments, the hydrogel is formed at room temperature. In some embodiments, the hydrogel is formed under a temperature of about 50° C. to about 90° C., about 60° C. to about 90° C., or about 60° C. to about 70° C. In some embodiments, the hydrogel is formed for about 1 h to about 4 h, about 2 h to about 4 h, or about 3 h to about 4 h.

In some embodiments, the step of fabricating the biosensing module further comprises attaching interdigitated electrodes to the hydrogel. The interdigitated electrodes can be S electrodes, M electrodes, or L electrodes as shown in FIG. 9.

In some embodiments, the step of fabricating the biosensing module further comprises adhering a protection layer in between the hydrogel and interdigitated electrodes. The protective layer can be a photoresistive coating such as SU-8.

The present invention also provides a method of monitoring a bacterial cell concentration of an in vitro sample from a subject, comprising:
a) contacting a biosensing module with the sample, the biosensing module being configured to output a signal indicative of presence of at least one biomolecule released by bacterial cells in the sample;
b) transmitting the signal from the biosensing module to a readout circuitry for wirelessly transmitting the signal to an external device.

The present invention also provides a method of monitoring a bacterial infection at a wound site, comprising:
a) contacting a biosensing module with the wound site, the biosensing module being configured to output a signal indicative of presence of at least one biomolecule released by bacterial cells at the wound site; and
b) transmitting the signal from the biosensing module to a readout circuitry for wirelessly transmitting the signal to an external device.

In this regard, the monitoring may be performed in vivo.

In some embodiments, the signal is produced by a change in dielectric permittivity, wherein the change in dielectric permittivity is converted into a change in capacitance before transmitting to the NFC module. In some embodiments, the change in dielectric permittivity is converted into a change in voltage before transmitting to the NFC module.

The present invention also provides a hydrogel comprising polynucleotide crosslinked with poly(ethylene glycol) diglycidyl ether (PEGDE). In this regard, the polynucleotide is covalently crosslinked to PEGDE to form the hydrogel.

The present invention also provides a method of fabricating a hydrogel comprising crosslinking polynucleotide with poly(ethylene glycol) diglycidyl ether (PEGDE). The hydrogel can be fabricated via 3D printing methods, or spin coating methods.

DNAgel synthesis and processability

To convert DNase activity into a smartphone-readable signal, we developed a DNAgel that meets processability requirements for integration into a bioelectronic sensor and exhibits a chemically-tunable dielectric permittivity over the radio-frequency spectrum. In contrast to prior work that used heating-cooling cycles to form physically-crosslinked DNA hydrogel, we used a chemical crosslinking strategy to provide increased stability in aqueous environments and capacity for functionalization. DNA strands were covalently crosslinked using poly(ethylene glycol) diglycidyl ether (PEGDE) to form a 3D hydrogel network (see Methods). The hydrophilic poly(ethylene glycol) component of PEGDE confers increased anti-dehydration and biocompatibility, while the two epoxide groups in PEGDE reacts with primary amine groups on the adenosine, guanine and cytosine nucleotide bases and bonds adjacent DNA strands (FIGS. 2A and B). This strategy yields a DNAgel with several advantageous properties: (i) less steric hindrance to subsequent permittivity engineering; (ii) fewer DNA strands (˜0.05 g dehydrated DNA in 1 mL of DNAgel) for increased sensitivity to DNase; and (iii) greater diffusion of reactive agents through the 3D network for more rapid response time. Importantly, the gelation reaction can be completed at room temperature without requiring heating or other harsh conditions. The ability of DNAgel to be gelated in situ enables it to be integrated into a wide range of bioelectronic interfaces. For instance, DNAgel precursor can be printed on either planar surface (FIGS. 2C and G) or curved contact lens (FIGS. 2D and H), or be molded into 3D macro (FIGS. 2E and I) and micro (FIGS. 2F and J) structures with a spatial resolution as fine as 10 μm.

### Selective Bioresponse and Anti-Dehydration Capability of DNAqel

The selective degradation of DNAgel by DNases associated with pathogenic bacteria provides the reporting mechanism for detection of an active wound infection. We first validated that DNAgel is selectively degraded by the opportunistic pathogen S. aureus ATCC 29213. DNAgel droplets were co-incubated with live neonatal human dermal fibroblasts (NHDFs) or S. aureus culture with comparable total cellular volume. Confocal fluorescence imaging showed that co-incubation with NHDFs (˜2×10⁴ cells) for over 1 hour resulted in negligible change in the droplet morphology (FIG. 3A). In contrast, co-incubation with S. aureus culture (˜7×10⁷ CFU) for 1 hour resulted in complete degradation of the DNAgel droplet (FIG. 3B). DNAgel degradation can be attributed to secretion of DNase (nuclease S7, also known as micrococcal nuclease) by S. aureus, as shown by immersing DNAgel (˜2.3×10⁸ m³ in volume) into a DNase solution (˜1 unit/μL) (FIG. 3C). 3D topography reconstructions show that DNAgel volume is reduced by 56% after 1 hour of immersion (FIG. 3D, E) and 68.3% after 1.5 hours. We further assessed the selective degradation of DNAgel by S. aureus compared to a panel of commensal skin-associated bacteria by incubating DNAgel with the respective sterile-filtered bacteria culture supernatant. Whereas DNAgel incubated with S. aureus over 24 h exhibited about 70% decrease in fluorescence intensity, DNAgel incubated with other skin commensal bacteria prevalent on the epidermis were not significantly degraded (FIG. 4).

We further evaluated the ability of the DNAgel to detect S. aureus infection in wound swabs collected from diabetic foot ulcer (DFU) patients. From a cohort of 18 DFU patients with available clinical microbiology reports, 3 patients were positive for S. aureus. We determined the total CFU for these samples and compared the S. aureus positive samples (n=3 patients) with patients harboring low bacteria colonization (<10⁴ CFU/cm²) as controls (n=5 patients). FIG. 3F and Table 1 show the change in fluorescence intensity of stained DNAgel co-incubated with the wound culture supernatant after 24 hours. Whereas DNAgel exposed to the S. aureus positive samples exhibited more than 52% decrease in fluorescence intensity in the test group, the fluorescence intensity of DNAgel exposed to control samples decreased by no more than 27%. These results suggest that DNAgel is degraded in the presence of S. aureus and the hydrolysis of DNAgel by other wound-colonizing microbes is minimal.

TABLE 1
Information about patient wound samples.

| | | | | | Fluorescence |
|---|---|---|---|---|---|
| Group | Patient ID | Age | Organisms | CFU/cm² | intensity |
| S. aureus | P1 | 69 | S. aureus | 42941 | 0.08 |
| group | P2 | 70 | S. aureus | 10181 | 0.39 |
| | | | Streptococcus agalactiae | | |
| | P3 | 56 | S. aureus | 274107 | 0.48 |
| | | | Peptostreptococcus anaerobius | | |
| Low CFU | P4 | 62 | Klebsiella pneumoniae | 0 | 0.73 |
| group | | | Prevotella spp. (moderate | | |
| | | | growth) | | |
| | P5 | 57 | E. coli | | |
| | | | Streptococcus anginosus | 0 | 0.91 |
| | | | E. faecalis | | |
| | | | Eikenella corrodens | | |
| | P6 | 64 | Streptococcus agalactiae | 198 | 0.94 |
| | | | Morganella morganii | | |
| | | | Citrobacter koseri | | |
| | P7 | 67 | Nil | 236 | 0.97 |
| | P8 | 56 | P. aeruginosa | 0 | 1.00 |

We also characterized the dehydration properties of DNAgel by placing 0.5 g of hydrogel in an opened centrifuge tube exposed to an environment with constant temperature (37° C.) and controlled relative humidity. The hydrogel maintains more than 80% weight at 70% relative humidity after 24 hours (FIG. 3G) which demonstrates a substantially longer dehydration time compared to common hydrogels such as x-carrageenan/polyacrylamide hydrogel that retain only 30% weight after exposure to similar conditions. This anti-dehydration property of DNAgel can be partially attributed to the hydrophilic poly(ethylene glycol) in the crosslinker. The dehydration time represents a lower bound for the lifetime of DNAgel because moisture is typically maintained in the wound environment by a wide range of wound dressings (FIG. 5).

Permittivity tunability and biocompatibility of DNAgel

Incorporating DNAgel with conductive dopants can increase sensitivity of the radio-frequency response to biological stimuli. We evaluated the tunability of dielectric permittivity of DNAgel by incorporating five different conductive dopants in the hydrogel network: poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), and silver nanowire (AgNW) (FIG. 6A, B). The biocompatibility of dopants alone was first examined by co-incubating DNAgels with NHDFs for 48 hours (FIG. 6C). Pristine DNAgel exhibited excellent biocompability with cell viability, assessed using trypan blue staining, similar to controls (FIG. 6D). The presence of conductive nanoparticles or polymers resulted in reduction of the cell viability, although dopant cytotoxicity was reduced after incorporation into DNAgel network (FIG. 7), which highlights the biocompatibility of the pristine DNAgel. We characterized the permittivity of doped DNAgels and found that AgNW can render a permittivity 1.47 times higher than that of pristine DNAgel within the tested bandwidth (1-200 MHz) (FIG. 6E). FIG. 6F shows a radar plot of the achievable range of biocompatibility and permittivity for the different dopants. For a viability threshold of 80% (FIG. 7), GOₓ and Ti₃C₂Tₓ MXene can achieve 29.5% and 28.6% increase in average dielectric permittivity, respectively. Nevertheless, we chose to use pristine DNAgel for this study due to its non-toxicity and sufficient permittivity for robust infection detection.

### Design and Optimization of the Capacitive Sensing Structure

To establish an interface for signal transduction, we designed a capacitive sensing structure consisting of an interdigitated electrode pattern on a flexible polyimide coated with DNAgel (FIG. 8A). Finite element simulations show the effect of the gap between electrodes d, the thickness of the DNAgel layer t₁, and thickness of the SU-8 layer t₂ on the sensor capacitance. As d is reduced from 350 to 50 μm, the intensity of the electric displacement field D fringing above the electrodes increases (FIG. 8B), which results in higher capacitance and sensitivity to the presence of the DNAgel layer. FIG. 8C displays an overview of these parameters by showing a stacked contour plot of sensor capacitance, as a function of (d, t₁, t₂). With a specific DNAgel thickness, a smaller electrode gap and thinner SU-8 can boost the capacitance induced by hydrogel. Based on the optimization result, we choose d=250 μm, t₁=1 mm and t₂=2 μm as the sensor parameters, which yields a capacitance of ˜0.15 pF/mm².

We numerically analyzed three electrode configurations (FIG. 8D, FIG. 9) and selected the design with ˜15 mm² active area for subsequent use, unless otherwise stated. The interdigitated structures were fabricated through a printing-and-etching process on a flexible printed circuit board (FPCB, see Methods). Cross-sectional images of the electrodes showed conformal coating of SU-8 over the copper (Cu) surface (FIG. 8E), which protects Cu electrodes and prevents potential cytotoxicity. A circuit comprising an LC tank and a half-wave rectifier was used to convert the capacitance signal into a voltage output Vₒᵤₜ (FIG. 8F). The resonant frequency of the LC tank was set as 13.56 MHz, in alignment with the frequency for NFC communication. We characterized the response of the sensing circuit by gradually decreasing the coverage area of DNAgel from ˜15 mm² to 0 mm² to mimic digestion by DNase. The circuit exhibited a change in the input impedance from Zᵢₙ=8.5+30.3j to Zᵢₙ=42.5+42.6j, resulting in a ˜0.7 MHz shift in the resonant frequency (FIG. 8G, H) and an ˜0.25 V increase in Vₒᵤₜ (FIG. 81, FIG. 10).

In Vitro and In Vivo Detection of S. aureus

The monitoring system integrates the DNAgel capacitive sensor and front-end circuit with a NFC module (FIG. 11) to enable battery-free and wireless data transmission through wound dressings. The wireless design utilizes two coils, the first for the LC biosensing module and the second for the NFC module, that have an optimized spacing between the coils of 3.5 mm (FIG. 12). FIG. 13 shows that the coil design achieves stable transmission to an external reader except under extreme misalignments. The monitoring system can be mounted on curved body surfaces by wound dressings, exhibiting negligible (±0.01 V) fluctuation in the readout signal when the bending angle is changed from 1800 to 600 (FIG. 14A). The wireless readout of the sensor is highly reproducible as DNAgel coverage is varied from 0 to 100% (FIG. 14B).

We evaluated the response of the monitoring system to S. aureus using the sterile-filtered culture supernatant. For in vitro experiments, the monitoring system was attached to gauze (1×1 cm²) permeated with filter-sterilized (0.22 μm filter) S. aureus culture supernatant at various concentrations at room temperature. The sensor was fixed by a Tegaderm film (3M) and wirelessly recorded via a smartphone over 48 hours. As shown in FIG. 14C, whereas control samples exposed to cell culture media resulted in no signal change after 24 hours, samples containing S. aureus culture supernatant produced a signal increase of 0.15 V for the 10⁵ CFU group and 0.38 V for the 10⁶ CFU group. FIG. 15 shows the dose-response curve of the sensor after 24 hours. The monitoring system produces a detectable signal when the amount of S. aureus exceeds 10⁵ CFU, which is at the lower end of clinical thresholds (10⁵ to 10⁶ CFU) widely used for laboratory diagnosis of infection. For amounts of S. aureus greater than 10⁷ CFU, the sensor response saturates at about 0.45 V due to complete degradation of DNAgel, thereby providing binary detection of infection. These results indicate that the sensor can detect secretory DNase activity when the amount of S. aureus approaches or exceeds thresholds for clinical infection.

We next demonstrated the ability of the monitoring system to detect wound infection in vivo using an acute wound model in mice. Full thickness excisional bilateral wounds (˜6 mm in diameter) through the panniculus carnosus were created on the dorsum with randomly assigned control and test sites (FIG. 14D). A piece of gauze with live S. aureus suspension (10⁵ or 10⁶ CFU) or sterile TSB was attached to the wound followed by placement and fixation of the sensor using adhesive dressings. Infected wounds with the monitoring system showed similar wound conditions as the uninfected control wounds after 24 hours (FIG. 14E). All sensors remained attached to the wound and the mice with the sensors did not show observable behavioural differences compared to controls (FIG. 14F). No obvious infection-related clinical signs, such as erythema, suppuration and friable granulation tissue were identified in both wounds. Wound culture at the experimental endpoint confirmed that S. aureus infection was established in the test groups (>10⁶ CFU/mL for both the 10⁵ and 10⁶ CFU groups) (FIG. 14G). Trace amounts of S. aureus (˜10⁵ CFU/mL) were also measured in the control (TSB) group, which can be attributed to S. aureus normally present on the skin and variations due to handling. Using a custom app, signals from the monitoring system were conveniently extracted by placing a smartphone in close proximity to the wound dressing. Wounds with live S. aureus (10⁵ and 10⁶ CFU) exhibited 0.4 V change in signal after 24 hours (FIG. 14H), triggering an infection alert on the smartphone (FIG. 14I).

### Threshold Studies

We performed additional in vitro experiments to study the response of the sensor for smaller (10⁴ CFU) and larger (10⁷ CFU) amounts of S. aureus. We used sterile-filtered (0.22 μm filter) culture supernatant as an equivalent substitute for live S. aureus suspension. The sensors were attached to gauze (1×1 cm²) spiked with S. aureus culture supernatant and fixed by Tegaderm film at room temperature. The recording was performed by a smartphone after 24 hours. FIG. 15 shows the resulting data and calibration curve with S. aureus from 0 CFU (TSB) to 10⁷ CFU. Signal change as a function of S. aureus CFU number when the monitoring system is exposed to S. aureus culture supernatant at room temperature after 24 h. Error bars show the mean±s.d. (n=5 data points). It can be seen that the presence of secretory DNase results in significant signal change of the monitoring system with CFU larger than 10⁴ (0.003 V): 0.15 V (10⁵ CFU), 0.38 V (10⁶ CFU), and 0.45 V (10⁷ CFU). Beyond 10⁷ CFU, the sensor signal saturates and essentially provides a binary response indicating infection. These results indicate that the sensor can quantitatively distinguish between amounts of S. aureus ranging from 10⁵ to 10⁷ CFU, corresponding to thresholds for clinically meaningful infection. However, we note that wound infections involve exponential growth in the amount of bacteria over a relatively short period of time.

### Stability of Hydrogel

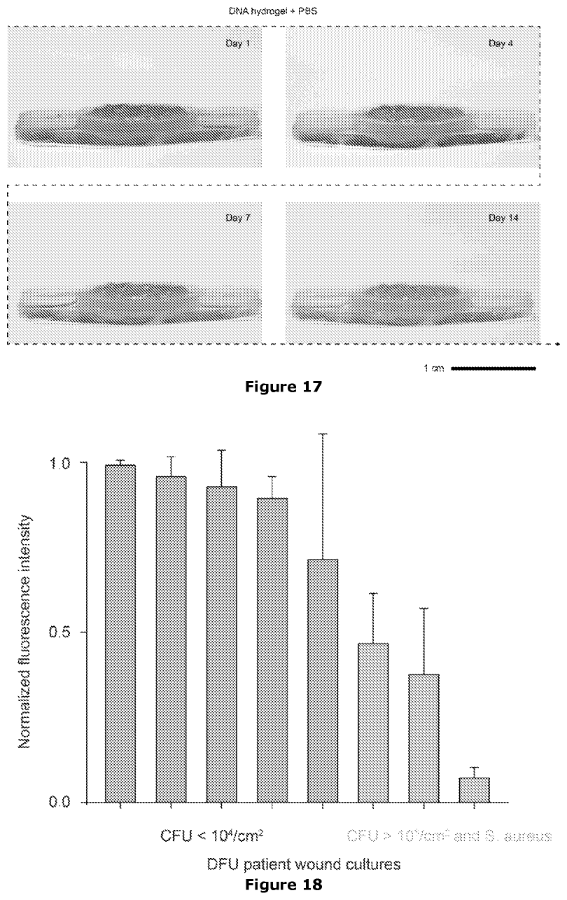

To rule out hydrolysis effect of hydrogel, we immersed DNAgel (a cylinder shape with 1 cm diameter and 1 mm thickness) into 1×PBS solution at 37° C. for 14 days. No obvious morphology change was observed (FIG. 17), which indicates that the DNAgel is stable in absence of enzymatic factors. FIG. 17 shows photos of DNAgel after immersion in PBS at 37° C. over 14 days. DNAgel is stained by Rhodamine B for clarity.

### Dose-Response Calibration Curve

We performed additional experiments to provide a dose-response calibration curve for the monitoring system, using smaller (10⁴ CFU) and larger (10⁷ CFU) amounts of S. aureus (FIG. 15). Briefly, sterile-filtered (0.22 μm filter) culture supernatant was used as an equivalent substitute for live S. aureus suspension. WINDOW sensors were attached to gauze (1×1 cm²), spiked with S. aureus culture supernatant, and fixed by Tegaderm film at room temperature. The recording was performed by a smartphone after 24 hours. FIG. 15 shows the resulting data and calibration curve with S. aureus from 0 CFU (TSB) to 10⁷ CFU. It can be seen that the presence of secretory DNase results in detectable signal change of WINDOW with CFU larger than 10⁵: 0.15 V (10⁵ CFU), 0.38 V (10⁶ CFU), and 0.45 V (10⁷ CFU). When applied to the 10⁴ CFU group, the sensor gives a 0.003 V signal change, which could be regarded as a lower limit of detection. The sensor signal saturates beyond 10⁷ such that it should be regarded as a binary detector for higher doses.

Evaluation with Patient Samples

We evaluated the ability of the DNAgel to selectively detect S. aureus infection in wound swabs collected from diabetic foot ulcer (DFU) patients. From a cohort of 18 DFU patients with available clinical microbiology reports, 3 patients were positive for S. aureus. We determined the total CFU for these samples and compared the S. aureus positive samples (n=3 patients) with patients harboring low bacteria colonization (<10⁴ CFU/cm²) as controls (n=5 patients). FIG. 18 shows the change in fluorescence intensity of stained DNAgel co-incubated with the wound culture supernatant after 24 hours. The fluorescence intensity of DNAgel co-incubated for 24 hours with culture supernatant of wound swab cultures from diabetic foot ulcer (DFU) patients is shown. Error bars show mean±s.d. (n=3 samples). DNAgel is stained using SYBR Gold. Whereas DNAgel exposed to the S. aureus positive samples exhibited more than 52% decrease in fluorescence intensity in the test group, the fluorescence intensity of DNAgel exposed to control samples decreased by no more than 27%. These results suggest that DNAgel is degraded in the presence of S. aureus and the hydrolysis of DNAgel by other wound-colonizing microbes is minimal.

### DISCUSSION

We have demonstrated a monitoring system, i.e. a flexible, wireless, and battery-free sensor based on DNAgel that can interface with wounds and detect infection. The sensor exploits material formulations, fabrication approaches, circuit layouts, and wireless techniques that collectively enable DNase activity associated with S. aureus virulence activity to be transduced into a wireless signal detectable by a smartphone. In vitro experiments establish that the sensor responds selectively to amounts of S. aureus near to thresholds for clinical infection (10⁶ CFU or more per gram of viable tissue) in both culture supernatant and in clinical wound exudates from diabetic foot ulcers. In vivo studies in a mouse wound model further demonstrate the utility of the sensor to detect clinically-relevant amounts of S. aureus when interfaced with wounds for 24 h.

Beyond detecting S. aureus, the monitoring system can be used to detect secreted DNases from other wound-associated pathogens such as P. aeruginosa and S. pyogenes. Although S. aureus and other bacteria commonly implicated in wound infections can be isolated from unaffected skin in many patients, they have dramatically lower abundance and expression of virulence factors on sites where the epidermis is not breached. Consequently, the presence of these and other skin commensal bacteria is not expected to significantly affect the signal reported by the sensor. It is believed that the wound microbial composition together with the monitoring system can be used to determine secreted DNase activity at wound sites which can serve as a general biomarker for infections. Possible effects of other virulence factors on the response of DNAgel can also be detected and monitored.

Because DNase activity is associated with many pathogenic bacteria, the sensing mechanism should be applicable to the detection of a broad range of other organisms implicated in clinical wound infections. Although the amount of S. aureus was selected according to a clinically-meaningful thresholds for infection (10⁶ CFU or more per gram of viable tissue), this threshold may vary with the organism and its interaction with surrounding microflora.

In this work, we demonstrated the bio-response of DNAgel to DNase secreted by S. aureus, an endonuclease that mainly cleaves on regions in the middle of target DNA strands, whereas this success did not exclude its possible response to other nucleases or pathogen. which may open avenues to sensing other pathogens beyond S. aureus (FIG. 16). It has been reported that the micrococcal nuclease prefers 5′-phosphodiester bonds in single-stranded DNA and is 30 times faster at A-T regions than at G-C. This fact implies that the network of our DNAgel consists of a large amount of single-stranded DNA and suggests a higher activity by increasing the single-stranded DNA ratio and using DNA strands with more A-T regions by sequence encoding techniques. Besides the response to nuclease, DNA hydrogel has been chemically modified to extend its response to diverse bio-stimuli. Beyond the enzymatic activity, these modifications may generate response to other pathogenic metabolites, improving the selectivity of DNAgel and making responsive drug delivery for synergistic wound management possible.

The functionality of DNAgel can be further expanded for clinical applications in wound monitoring. Depending on the wetness of the wound environment, dehydration of DNAgel can limit time duration over which the sensor is effective. For example, bonding a thin elastomer film to hydrogel can greatly increase anti-dehydration and adding microfluidic structures can constrain dehydration while helping to dissipate liquified gel after enzymatic degradation. Using CRISPR-associated nucleases, DNAgel degradation actuated by specific RNA inputs can also be demonstrated, which could be exploited as a detection mechanism for pathogens not associated with DNase. The sensitivity of DNAgel is presently limited by the cytotoxicity of conductive dopants. Biocompatible dopants, such as coated inert gold particles, could also yield approaches to increase sensitivity.

Clinically, the sensor could be embedded in wound dressing to enable patients to monitor their wounds between clinical assessments and seek appropriate intervention in the event that infection is detected. For example, in addition to detecting infection, quantitative assessment of infection severity could be valuable in helping to determine the appropriate treatment at the point of care. In this regard, existing sensors for measuring wound temperature, moisture, and pH as well as specific biomarkers could be integrated with the device to provide multiplexed analysis. Alternative wireless technologies could also be used to enable passive streaming of data from the sensor without requiring patients to bring a smartphone in proximity to the wound. This mode of operation requires the sensor to have a power supply, which may be addressed using a combination of solutions for energy storage, wireless charging, and energy harvesting. In conclusion, our wireless wound biosensor represents a step in facilitating and empowering personalized monitoring of wound infection to ensure that wound patients can receive prompt treatment and clinical care.

In conclusion, inspired by the NETs-DNase interaction, we developed a wireless wound infection sensing tag functionalized by DNAgel, as an effective bio-electrical signal transducer. The 3D network of DNAgel provided adequate space for reactive DNase diffusion and permittivity engineering. Quantitative fluorescent imaging corroborated that our DNAgel has selective response to S. aureus and its secreted DNase, rather than normal human cells. Its processability enabled intimate coating over an interdigitated capacitive biosensor capable of converting the enzymatic activity to electrical signal. By cascading the biosensor to a NFC module, we got the WiTag and demonstrated the feasibility of S. aureus monitoring in vitro and in vivo. Our DNAgel and its successful integration with wireless sensing platform may open avenues to new wound management approaches and better wound healing outcomes. Also, the investigation on other bio-responsive hydrogels will enable unprecedented skin-interfaced or implantable wireless bioelectronics, enabling the prompt detection of wound infections and the pathogen-responsive drug delivery at wound sites.

The present invention has the following advantages:

| | |
|---|---|
| Capacitance sensing mode | Changes in capacitance due to change in |
| | wound conditions is converted into a |
| | change in resonant frequency and signal |
| | voltage. |
| | The capacitance sensing mode allows |
| | easy interfacing with wireless readout |
| | setup. |
| Flexible interdigitated capacitive | Interdigitated capacitive biosensor |
| biosensor | provides an entity to realize capacitance |
| | sensing mode. |
| | Flexible feature greatly benefit skin- |
| | interfaced sensing as well as integration |
| | with commercial wound dressings. |
| Bioresponsive DNA hydrogel | DNA hydrogel has tissue-compatible |
| | modulus, excellent processability, |
| | tunable permittivity, anti-dehydration |
| | and good selectivity to pathogenic |
| | activity. |
| | The DNA hydrogel serves as an effective |
| | pathogen indicator and a bio-electrical |
| | signal transducer for biosensor. |
| Near field communication technique | Eliminate the use of battery or wired-in |
| | scheme |
| | Prevent the eavesdropping of personal |
| | information by other receivers |
| Smartphone-based signal readout | Send and receive information via daily |
| | smartphone with a user-friendly interface |

The present invention can be applied in:
a) In-situ real-time monitoring. Wireless sensing tag can continuously monitor the wound site on-demand without disturbing the wound dressing and healing process. One of the applications of the invention is to monitor post-surgical or traumatic wound infections.
b) Avoid symptoms of complications. Post-surgical complications related to infection are usually realized with serious symptoms at very late stage. This leads to invasive and expensive clinical treatment. Our invention solves such clinical pain point, as the infection can be wirelessly detected in real time.
c) Remote sensing. This invention allows remote sensing of wound sites, solving the visual hindrance by wound dressings over wounds.
d) Battery-free communication. This invention is based on a smartphone-driven wireless sensing scheme, which enables long-term and easy deployment.

### Materials and Methods

### DNAgel Synthesis

DNA strands were covalently crosslinked using poly(ethylene glycol) diglycidyl ether (PEGDE) to form a 3D hydrogel network. The hydrophilic poly(ethylene glycol) component of PEGDE confers increased anti-dehydration and biocompatibility, while the two epoxide groups in PEGDE reacts with primary amine groups on the adenosine, guanine and cytosine nucleotide bases and bonds adjacent DNA strands. Specifically, DNAgel precursor was prepared by dissolving 10 wt % deoxyribonucleic acid sodium salt (smDNA) in 4.0 mM NaBr solution at room temperature. 2.5 wt % crosslinker, poly(ethylene glycol) diglycidyl ether (PEGDE, Mn=500), was uniformly mixed with the precursor. 0.5 wt % N,N,N′,N′-Tetramethylethylenediamine (TMEDA), as the catalyst, was further mixed with the hydrogel precursor. The precursor can be printed onto planar/curved surface or casted into macro/micro mold and kept under 90% relative humidity for 48 hours to complete the cross-linking reaction. To speed up the reaction, the precursor can be transferred into a sealed centrifuge tube and immersed in a water bath at 85° C. for 2 h to complete the gelation. After gelation, the prepared DNAgel was thoroughly rinsed by deionised (DI) water to remove unreacted chemicals. All DI water used in the experiment was from Barnstead Nanopure ultrapure water system (Thermo Fisher Scientific).

Bio-response and anti-dehydration of DNAqel

Wound isolate S. aureus ATCC 29213 (SA29213) from a streak plate was inoculated into 10 mL sterilized tryptic soy broth (TSB, Sigma-Aldrich) and allowed to grow overnight at 37° C. at 200 rpm. The colony-forming unit (CFU) of S. aureus was characterized by optical density (OD) using a spectrometer. Neonatal human dermal fibroblasts (NHDFs) were incubated in the medium composed of Dulbecco's Modified Eagle's medium (DMEM, Thermo Fisher Scientific)+10% fetal bovine serum (FBS)+1% penicillin-streptomycin at 37° C., in a humidified atmosphere (5% CO₂). 3000 units/mL DNase (Nuclease S7, Aldrich) stock was prepared using 0.5 mM CaCl₂) solution (sterilized by 0.22 μm filter). All stocks were further diluted by corresponding media before imaging. To make a fair evaluation of selectivity, the total cellular numbers of NHDFs and S. aureus were determined based on an assumption that the effective metabolites produced by NHDFs and S. aureus, for instance DNase, are equivalent per unit cell volume. Given the volume of fibroblast ˜2000 m3 and S. aureus ˜0.52 μm3 (58), the total cellular volume of NHDFs (˜2×10⁴ cells, 4×10⁷ μm³) and S. aureus (˜7×10⁷ CFU, 3.7×10⁷ μm³) are comparable, therefore confirming the selective response of DNAgel.

For the imaging, DNAgel samples were transferred into a chambered borosilicate coverglass system (Lab-Tek, Thermo Scientific). Fluorescent images were acquired by a confocal microscope (Zeiss LSM 710) in Z-Stack mode with controlled ambient by Zeiss incubation system. 3D topography of fluorescent images was reconstructed by Imaris package (Oxford Instrument). A bacterial viability stain (LIVE/DEAD BacLight Bacterial Viability Kit, Invitrogen, Thermo Fisher Scientific) was used for the S. aureus, a plasma membrane stain (CellMask, Invitrogen, Thermo Fisher Scientific) for NHDFs, and a fluorescent stain (NucBlue, Invitrogen, Thermo Fisher Scientific) for the DNAgel and NHDFs nuclei. The de-hydration tests were performed in a chamber (SH-262, ESPEC) with controlled temperature and relative humidity.

In Vitro Degradation of DNAqel by S. aureus and Skin Commensal Bacteria Strains

DNAgel was prepared as described above. SYBR Gold Nucleic Acid Stain (Invitrogen, Thermo Fisher Scientific) was added to the gel precursor immediately after the addition of TMEDA and mixed uniformly. 25 μL precursor drops were placed onto the lids of 150 mm tissue-culture treated dishes (Corning), sealed with Parafilm and kept away from light for 48 hours for complete cross-linking. The crosslinked DNAgel was then washed thoroughly with ultrapure water (Merck Millipore).

Cultures of ATCC bacterial strains were grown for 24 hours in TSB at 37° C. The optical density at 600 nm (OD600) of each culture was noted. The cultures were then centrifuged (5,000×g, 10 min, 4° C.), sterile-filtered (0.22 μm filter) and stored at −20° C. until needed. 25 μL of sterile-filtered culture supernatant was added to each DNAgel drop, and incubated at 37° C. for 24 hours. Positive controls (DNAse I, Zymo Research) and negative controls (sterile TSB) were also set up.

DNAgel drops were imaged immediately after addition of culture supernatants and after 24 hours incubation, using the Gel Doc™ EZ Imager and UV Tray (Bio-Rad). The change in size and fluorescence intensity of each DNAgel drop was quantified with ImageJ. Experiments were performed in technical triplicates.

### DNAqel Response to Patient Wound Samples

Wound sampling from diabetic foot ulcer (DFU) patients was approved by St Luke Hospital's Institutional Review Board (IRB-02-2019-08-28) and all subjects provided written consent before participation. The inclusion criteria for this study comprised: 1) male/female individuals >21 years old who have received a clinical diagnosis of diabetes, 2) able to provide consent, and 3) have one or more diabetic foot ulcers present on the lower limb. Patients who were involved in other interventional clinical trials were excluded from this study. The wounds were cleansed with sterile water before sampling, and one sterile Levine swab was used to collect the wound fluid and microbes from each patient prior to debridement. 500 μL of 50 mM Tris pH 6 with 5 mM CaCl₂) was added to each swab on the same day the swab was collected, and vortexed for 30 sec. 100 μL of the sample was mixed with 400 μL of Tryptic Soy Broth (TSB) containing 15% glycerol and stored at −80° C. in aliquots until further processing. For 18 patient samples with available clinical microbiology reports, we determined the colony-forming unit (CFU) count. 10 μL of the sample was thawed, diluted and plated onto TSB agar plates and incubated for 48 hours before a manual count was done (in triplicates). Samples with S. aureus (3 patients, >10⁴ CFU/cm²) and low microbial colonization (5 patients, without S. aureus, <10⁴ CFU/cm²) were selected for DNAgel test. To assess DNase hydrogel degradation, 10 μL of each sample was added to 4 mL of TSB and incubated for 24 hours at 37° C. with shaking at 200 rpm. The culture supernatant was obtained by centrifuging the culture at 5000 rpm for 5 minutes and then filtered with 0.22 μM filters. The DNA hydrogel degradation assay was performed as per the cultured bacteria strains above.

### DNAqel Doping and Characterization

Ti₃C₂Tₓ MXene nanosheets were prepared according to the literature. 1.0 g of lithium fluoride (LiF, Sigma-Aldrich, BioUltra, 99.0%) was added to 6.0 M hydrochloric acid (HCl, Sigma-Aldrich, ACS reagent, 37%) solution (20 mL) under vigorous stirring. After the dissolution of LiF, 1.0 g of Ti₃AlC₂ powder (Tongrun Info Technology Co. Ltd) was added slowly into the HF-containing solution, the mixture was then kept at 35° C. for 24 hours. Thereafter, the solid residue was washed with deionized water several times until the pH value increased to ca. 7.0. Subsequently, the washed residue was added into 100 mL of deionized water (Millipore), ultrasonicated for 1 hour under N2 atmosphere, and centrifuged at 3,000 r.p.m. for 30 minutes. The supernatant was collected as the suspension of Ti₃C₂Tₓ MXene nanosheets.

0.2 wt % Ti₃C₂Tₓ MXene, graphene oxide (GOₓ, Timesnano), poly(3,4-ethylenedioxythiophene)-poly(styrene sulfonate) (PEDOT:PSS, Clevios PH1000, Heraeus), single-wall carbon nanotubes (SWCNTs, Timesnano), and silver nanowires (50 nm in diameter, Kechuang Advanced Materials) were doped into hydrogel precursor, respectively. The gelation was completed following the protocol of undoped hydrogel. After the gelation, the permittivity of hydrogels was obtained using a dielectric probe (85070E, Keysight) and a vector network analyzer (N9923A FieldFox, Keysight) after calibration by deionized water.

To evaluate the cytotoxicity of dopants, ˜1 μL DNAgel and dopants (1 wt %) were spiked into 50 μL NHDFs (˜4×10⁴ cells) and incubated for 48 hours. The NHDFs were then observed under a microscope (Nikon Eclipse Ti2 microscope) after treatment by LIVE/DEAD™ Cell Imaging Kit (Invitrogen, Thermo Fisher Scientific). For viability results, 50 μL NHDFs (˜4×10⁴ cells) were incubated with ˜1 μl DNAgel and dopants (1 wt %) for 48 hours and tested through trypan blue staining and standard MTT assay.

### Monitoring System Design and Fabrication

Interdigital capacitive sensing electrodes were modeled by using more than 2.48×10⁶ tetrahedrons and simulated by the finite-difference time-domain method (CST Microwave Studio, Dassault Systems) to evaluate the capacitive sensing performance. The optimization was realized through systematically sweeping key geometrical parameters. Mutual coupling analysis was conducted by CST with circuit layout from Altium Designer.

The interdigitated electrodes were fabricated by printing (ColorQube 8880, Fuji Xerox) the traces (FIG. 9) on a copper-polyimide substrate (18-μm-thick copper, 25-μm-thick polyimide layer, DuPont). After baking at 70° C. for 10 min, the printed substrate was etched using H₂O₂ and HCl and cleaned by immersion in hexane and ethanol to yield the patterned traces. SU-8 was coated and UV cross-linked over the sensor surface as protection layer with a thickness of ˜2 μm. A crescent silicone pillar (˜1 mm in thickness) was added onto the capacitive sensor for mechanical support, followed by DNAgel functionalization (˜1 mm in thickness) of the active region of the sensor. The circuit diagram for NFC module and the electronic components involved can be found in FIG. 11.

### In Vitro Evaluation of Monitoring System

Culture supernatant of SA29213 was used for in vitro test. SA29213 was grown overnight on tryptic soy agar (TSA, Sigma-Aldrich). Single colony of SA29213 was inoculated in tryptic soy broth (TSB, Sigma-Aldrich) and allowed to grow to OD600 0.8 at 37° C. TSB was then inoculated (OD600 0.01) and cultured at 37° C. overnight. Overnight culture, with tested CFU number, was clarified via centrifugation (3,000×g, 30 min, 4° C.), sterile-filtered (0.22 μm filter) and stored at −20° C. until needed. For in vitro experiment, culture supernatant was diluted by TSB, as an equivalent substitute for live S. aureus suspension with effective secretory DNase. The hydrogel coverage response was recorded by a mixed domain oscilloscope (MD03012, Tektronix) and a vector network analyzer (N9923A FieldFox, Keysight).

### In Vivo Evaluation of Monitoring System

Male C57 black 6 inbred mice (C57BL/6) between 8-10 weeks of age and 25-30 g of weight were used. Mice were provided by in-house colony by LKC medicine animal facility. The skin on the back of the mice was prepared by shaving and applying depilatory cream (Nair). The injury site was then wiped three times with 70% ethanol. Surgery was performed under inhaled isoflurane (2% to 5%), and depth of anesthesia was checked by testing pedal reflex. Buprenorphine (1.5 mg/kg) was injected subcutaneously before wounding for sustained pain relief. Full thickness excisional wounds through the panniculus carnosus were achieved by lifting the back skin of the mice from the dorsum and making an incision with a 6-mm biopsy punch (Acuderm Inc.). The two bilateral wounds equidistant from the midline and spaced either side of the dorsum were randomly assigned as the control wound or the test wound for WINDOW application.

Mice were divided into three groups where test wounds were applied with either TSB or live bacteria suspension of SA29213 at 105 or 106 CFU (n=2 mice per group). Overnight S. aureus culture was diluted with TSB to achieve target CFU numbers for the experiment. Gauze was overlaid onto the wound and 20 μL bacteria suspension/TSB applied directly onto the gauze and wound. DNAgel-functionalized WINDOW was then placed onto the gauze and fixed by a small piece of Tegaderm film (3M). The whole back of the mice was then covered with a OPSITE dressing (Smith and Nephew) to ensure both the monitoring system and gauze remained in place. A mobile phone with a custom app was used to record the signal, 0, 1, 4 and 24 hours after monitoring system attachment. To quantify the amount of bacteria 24 hours post-wounding, mice skin surrounding the wound was sampled (approximately 1 cm×1 cm) and placed in pre-weighed 2 mL microcentrifuge tubes containing 1 mL sterile PBS. The mice skin sample was weighed and sonicated in a chilled sonicator (Elmasonic S 30 H, Elma Schmidbauer GmbH, Germany) to dissociate adherent bacteria (37 kHz, 10 min per cycle, 3 cycles, 1 min vortex after each cycle). Bacteria in each sample was enumerated via CFU counting and normalized by sample weight. All experiments conformed to the Guide for the Care and Use of Laboratory Animals published by the National Institutes of Health, USA, and protocol approved by the Institutional Animal Care and Use Committee (IACUC) of the Animal Research Facility of Nanyang Technological University, with consideration to ethical use and animal welfare.

### Circuit Design of Monitoring System

Our wireless sensing tag is composed of two modules: (1) a DNAgel functionalized biosensing module for bacteria detection and (2) a NFC module for wireless transmission. The selected NFC module (RF430FRL152H, Texas Instrument) is a low power NFC with a built-in MCU (MSP430 series) and analog-to-digital converter (ADC) at sampling rate ˜1 Hz. The sensor module is designed as a LC resonant circuit with a resonance frequency at 13.56 MHz to match the operation frequency of the NFC system. The sensor and NFC modules are powered by inductive (magnetic field) coupling with the smartphone reader.

Due to the limitation of the ADC performance (0.9 V maximum input voltage and 1 Hz sampling frequency), the change of signal intensity at 13.56 MHz cannot be directly captured by the ADC. Hence, we incorporated a half-wave-rectified circuit and a voltage divider to convert the AC signal to a semi-DC signal. Upon exposure to pathogenic bacteria, DNAgel is gradually digested, altering the permittivity and capacitance of the interdigitated sensing electrodes. The change of capacitance further shifts the resonance frequency of LC circuit and signal intensity at 13.56 MHz.

To conceptually prove the sensing capability of our tag, we simplified the circuit (FIG. 10A). The power source of the circuit is modeled as an induced AC voltage source at the coil. The coupling between the sensor and the reader is neglected due to the weak coupling (FIG. 12). The sensor is modeled as a variable capacitor and an equivalent series resistor (ESR) that represents the dielectric loss in the sensor. The rectifier, voltage divider circuit, and the ADC input impedance are simplified as an impedance Z.

Given these conditions, the gain of the system is solved from the Kirchhoff Laws and can be represented as,

$$Gain = \frac{{V}_{o}}{{V}_{i}} = {[ 1 + ( j ω L + {R}_{L} ) ( \frac{1}{Z} + j ω {C}_{res} + \frac{tan δ + \frac{j}{{( ω {C}_{s} )}^{2}}}{ω {C}_{sensor} ( tan {δ}^{2} + \frac{j}{{( ω {C}_{s} )}^{4}} )} ) ]}^{- 1} ( S 1 )$$

where φ, R_{L}, L, Cᵣₑₛ, Cₛ, and tan δ represent the angular frequency, coil resistance, coil inductance, resonance tuning capacitor, sensor capacitance and dielectric loss of sensor, respectively.

If we assume a negligible loss in the sensor, we can further simplify the equation as:

$$Gain = \frac{{V}_{o}}{{V}_{i}} = {[ 1 + ( j ω L + {R}_{L} ) ( \frac{1}{Z} + j ω ( {C}_{res} + {C}_{s} ) ) ]}^{- 1} \qquad ( S 2 )$$

From the circuit model, the response of the gain as a function of the sensor capacitance is shown in FIG. 10B. The change of the capacitance results in the change of the signal gain, which confirms our circuit as a bio-electrical signal transducer.

### NFC Technique

Near-field communication (NFC) is a short-range (typically up to 4 cm) wireless technology found in most modern smartphones that is widely used for contactless payment and other applications. NFC is derived from radio-frequency identification (RFID) technology and allows a reader to communicate with passive, battery-free electronic tags for identification, tracking and sensing. NFC operates in the 13.56 MHz industrial, scientific, and medical (ISM) band, and implements power and data transfer through magnetic inductive coupling between the reader and the tag with data rates ranging from 106 to 424 kbit/s. NFC has recently been used for a variety of biosensing applications, including spinal posture monitoring, neonatal intensive care, and sweat analysis. A key advantage of NFC is that it enables the biosensor to have a small footprint and thin profile because it enables the data readout to be performed by the smartphone in a wireless and battery-free manner. In addition, the short communication distance can mitigate data privacy concerns during operation owing to the reduced risk of eavesdropping by third parties.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A wound monitoring system for monitoring a bacterial infection at a wound site, comprising:
a) a biosensing module that is contactable with the wound site, the biosensing module being configured to output a signal indicative of presence of at least one biomolecule released by bacterial cells at the wound site; and
b) readout circuitry coupled to the biosensing module for wirelessly transmitting the signal to an external device;
wherein the signal is produced by a change in dielectric permittivity of the biosensing module.

2. The wound monitoring system according to claim 1, wherein the signal is produced by a change in dielectric permittivity of the biosensing module due to degradation of the biosensing module.

3. The wound monitoring system according to claim 1, wherein the biosensing module comprises a hydrogel, the hydrogel comprising polynucleotide crosslinked with poly(ethylene glycol) diglycidyl ether (PEGDE).

4. The wound monitoring system according to claim 3, wherein the polynucleotide is single stranded DNA and/or single stranded RNA.

5. The wound monitoring system according to claim 3, wherein a concentration of polynucleotide in the hydrogel is about 0.02 g/mL to about 0.1 m/gmL, and wherein a concentration of PEGDE in the hydrogel is about 0.01 g/mL to about 0.05 g/mL.

6. The wound monitoring system according to claim 3, wherein the hydrogel further comprises a dopant selected from poly(3,4-ethylenedioxythiophene)-poly(styrene sulfonate) (PEDOT:PSS), Ti₃C₂Tₓ MXene, graphene oxide (GOₓ), single wall carbon nanotube (SWCNT), silver nanowire (AgNW), or a combination thereof.

7. The wound monitoring system according to claim 6, wherein the dopant has a concentration of about 0.1 wt/wt % to about 1 wt/wt % relative to the hydrogel.

8. The wound monitoring system according to claim 3, wherein the hydrogel retains more than 80% of its weight at 70% relative humidity after 24 hours, and/or wherein the hydrogel has a thickness of about 0.4 mm to about 10 mm.

9. The wound monitoring system according to claim 1, wherein the at least one biomolecule is a nuclease.

10. The wound monitoring system according to claim 1, wherein the signal indicative of presence of at least one biomolecule released by bacterial cells at the wound site is a change in a dielectric permittivity of the biosensing module of about 0.1 F/m to about 20 F/m.

11. The wound monitoring system according to claim 1, wherein the biosensing module comprises a capacitive sensor for converting the change in dielectric permittivity into a change in capacitance, wherein the capacitive sensor comprises interdigitated electrodes having an inter-electrode spacing between about 30 μm and about 400 μm.

12. The wound monitoring system according to claim 11, wherein the capacitance is of about 0.4 pF to about 2.5 pF.

13. The wound monitoring system according to claim 11, wherein the change in capacitance is convertible into a change in voltage.

14. The wound monitoring system according to claim 1, wherein the readout circuitry comprises a near field communication (NFC) antenna.

15. The wound monitoring system according to claim 1, wherein the biosensing module is electrically connected with the readout circuitry, wherein a distance between the biosensing module and the readout circuitry is between about 2 mm to about 10 mm.

16. A method of fabricating a wound monitoring system for monitoring a bacterial infection at the wound, comprising:
a) configuring a biosensing module to output a signal indicative of a presence of at least one biomolecule released by bacterial cells at the wound site when the biosensing module is contacted with the wound site; and
b) coupling a readout circuitry to the biosensing module for wirelessly transmitting the signal to an external device;
wherein the signal is produced by a change in dielectric permittivity of the biosensing module.

17. The method according to claim 16, further comprising a step of fabricating the biosensing module, the step comprising crosslinking polynucleotide with poly(ethylene glycol) diglycidyl ether (PEGDE) in order to form a hydrogel for contacting with the wound site.

18. The method according to claim 17, wherein the step of fabricating the biosensing module further comprises attaching interdigitated electrodes to the hydrogel.

19. The method according to claim 16, wherein the step of fabricating the biosensing module further comprises adhering a protection layer in between the hydrogel and interdigitated electrodes.

20. A method of monitoring a bacterial cell concentration of an in vitro sample from a subject or a bacterial infection at a wound of a subject, comprising:
a) contacting a biosensing module with the sample or the wound, the biosensing module being configured to output a signal indicative of presence of at least one biomolecule released by bacterial cells in the sample or at the wound site;
b) transmitting the signal from the biosensing module to a readout circuitry for wirelessly transmitting the signal to an external device;
wherein the signal is produced by a change in dielectric permittivity of the biosensing module.

21. (canceled)
